(19) United States
(12) Patent Application Publication
(10) Pub. No.: US 2025/0258204 A1
(43) Pub. Date: Aug. 14, 2025

(54) DETECTION SYSTEMS

(71) Applicant: Illumagear, Inc., Seattle, WA (US)

(72) Inventors: Matt Squires, Atlanta, GA (US); Jedediah Joaquin McCann, Portland, OR (US); Joshua Robert Buesseler, Seattle, WA (US); Kyle Visner, Seattle, WA (US); Ben Salinas, Austin, TX (US); Chester Macklin, Austin, TX (US); George Harris, Austin, TX (US); John Maxwell Baker, Seattle, WA (US)

(21) Appl. No.: 18/856,443

(22) PCT Filed: Apr. 17, 2023
(86) PCT No.: PCT/US2023/018864

§ 371 Date: Oct. 11, 2024

(51) Int. Cl. G01R 19/145; A61B 5/00; A61B 5/11; G06F 1/16

(52) U.S. Cl. CPC G01R 19/145; A61B 5/1117; A61B 5/6803; A61B 5/742; A61B 5/746; A61B 2503/20; A61B 2562/04; G06F 1/163

(57) ABSTRACT

A detection system includes a detector unit capable of detecting whether a current is active in an environment in a vicinity of a worker or other user, whether a voltage is active in the environment in the vicinity of the worker or other user, and/or whether a fall event associated with the worker or other user has occurred. The detector unit includes a status indicator with at least one characteristic of the status indicator changing in response to detection of one or more such events as a method of signaling or warning the worker or other user regarding the detected event. The detector unit may be independently wearable or carryable by the worker or other user and/or may interface with a receiver coupleable to equipment of the worker or other user. The detector units may be in communication with each other and with other devices within a worksite safety system.

### BACKGROUND

### Technical Field

The present disclosure generally relates to detection systems, such as, for example, detection units configured to detect occupational risks and other events in a vicinity of industry workers or other users.

The disclosure also generally relates to techniques for communication between signaling devices and a broader computing network and/or remote devices for dynamically providing alerts and warnings of the detected occupational risks to industry workers and other users.

### Description of the Related Art

It is well-known that workers in certain industries are routinely exposed to potentially life-threatening occupational hazards. For example, electricians, line workers, and construction workers, among others, may be exposed to dangerously high voltage and currents as part of their ordinary job responsibilities. These workers are also at risk of fall events, which can include a fall from an elevated position that could produce serious and potentially life-threating injuries as well as a fall that is a result of an injury from, or contact with, another source (i.e., falling debris or machinery that knocks a worker to the ground quickly). In response, various devices have been proposed to reduce the risk of accidents due to these hazards at jobsites. For example, conventional voltage and current detectors may be used to determine whether a wire or cable has an active voltage and current, respectively. In addition, workers operating at elevated positions may be required to wear a harness and a safety line for certain tasks to reduce the risk of a fall event. Certain devices capable of detecting fall events have also been proposed. However, these are incomplete solutions that have a number of disadvantages.

In particular, conventional voltage and current detectors typically provide a point in time measurement, such as whether a wire has voltage or current at a single point of time. Naturally, this creates risks for workers if voltage and current are introduced at a later point in time, unbeknownst to the worker. In addition, these solutions are not able to sense whether the voltage or current in a line is beyond a certain threshold over time. For example, a worker may be aware that a line has a certain voltage or current and may be able to work safely in the area with the known voltage or current, but is not alerted if the voltage or current changes beyond the worker's expectation. Moreover, these voltage and current detectors may not sufficiently identify risks in areas with multiple lines with different voltages and currents, as conventional voltage and current detectors are limited to sensing voltage or current in one specific line at a single point in time. There are other disadvantages as well, such as power issues with rechargeable detector devices where a worker forgets to charge or replace the batteries of the device, thus rendering the device inoperative and ineffective at preventing occupational risks.

With respect to potential fall events, harnesses and safety lines have proven effective at preventing falls from elevation when they are appropriately used. However, it is known that workers do not always follow safety rules with respect to harnesses and safety lines because such lines hinder the workers' movements. Further, it is possible for fall events to occur even with appropriate use of a harness and safety line, but such fall events may not be reported to other onsite staff and workers, such as supervisory and medical staff. Moreover, a conventional harness and safety line does not anticipate or prevent all the types of fall events that can occur in a work environment. For example, a worker may be injured by falling debris and fall to the ground quickly or be knocked over by machinery, both of which are types of a fall event that cannot be prevented with a harness and safety line, but that are nonetheless highly relevant events for worker safety and for other onsite staff and personnel to be aware of. While some devices have been proposed to detect fall events, current solutions may not adequately provide an alert for a fall event or otherwise notify onsite staff so that an appropriate response can be carried out. In other words, the signaling function of certain devices capable of detecting falls is inadequate. In addition, devices that may be capable of detecting fall events may not be able to detect both falls from an elevated position as well as falls associated with a worker or other user being knocked over, but rather, are limited to detecting only specific types of falls.

As a result, it would be advantageous to have detection system, devices, and methods that overcome the disadvantages of known solutions.

### BRIEF SUMMARY

Embodiments described herein provide detection devices, systems, and methods that are particularly well adapted to determining active voltage and electrical currents in an environment in a vicinity of a worker or other user of the detection system in real time. The detection systems may also include an accelerometer to determine whether a fall event has occurred. These devices, systems, and methods are particularly well adapted for workers at risk of electrical shock and fall events, such as, for example, workers in the construction industry, electrical industry, mining industry and other hazardous or hostile environments. The detection device, systems, and methods generally utilize a detector unit that may be provided in one of a number of different form factors. For example, the detector unit may be in the form of a functional detector cartridge with at least one status indicator, such as a lighting element, a speaker and/or buzzer, or other devices configured to deliver audible, visual, and/or haptic warnings to a user. The cartridge further includes sensors and a controller in communication with the status indicator and the sensors. The controller includes at least one processor and a memory that stores computer-readable instructions that, when executed by the at least one processor, cause the sensors of the detector unit to detect whether a voltage field is active in an environment in a vicinity of the user, whether a current is active in the environment in the vicinity of the user, and whether a fall event associated with the user of the detector unit has occurred in real time. In response to a detected event, the controller of the detector unit may execute instructions to change at least one characteristic of the status indicator (i.e., issue an audible, visual, and/or haptic alert or warning) and also send a communication to one or more remote computing devices to notify other users within a broader safety system or network.

The detector unit and associated controller are further configured to determine whether voltage or current are present above selected thresholds. Thus, for example, a comparatively low voltage or current that is known to the worker may not result in an alert issued by the detector unit, but the worker is alerted, as above, if the voltage or current increases beyond a selected threshold value. This may occur, for example, if the worker moves closer to a source of the voltage or current. In other instances, the selected threshold value may be exceeded when a source of the voltage or current is activated from a deactivated state. In some non-limiting examples, the detector unit may have a threshold adaptive mode where initial warnings are silenced, disabled or not reissued until a further positive change in the voltage or current is detected, which may arise, for example, when the worker or other user moves closer to the source of the voltage or current and/or when the magnitude of the detected voltage or current increases for another reason (e.g., another line is activated), or until the detector unit determines a percentage change in a magnitude of the detected voltage or current over a selected period of time. The detector unit may further be associated with a lighting device, such as a wearable or carryable lighting device. In some examples, the lighting device may provide power to the detector unit to increase the overall battery life of the detector unit and reduce the likelihood that the detector unit will exhaust its battery during routine use. The detector unit may also be in communication with the lighting device to enable the detector unit to receive one or more signals from the lighting device and/or to transmit one or more signals to the lighting device. Thus, for example, the detector unit may send a signal to the lighting device to change at least one characteristic of the lighting device, such as a lighting scheme among others, in response to a detected event.

The detector unit may be one of a number of detector units active at one or more worksites, and the detector units at each worksite or across sites may be in communication with each other as well as with a personal mobile device and a broader network to provide additional functionality, such as, for example, to group detector units for a work group in a single communication channel so that detected events may be communicated to the group. The detector units may also send data to a server regarding a detected event, which in turn, can issue an SMS or text message alert, or otherwise notify supervising or other selected staff regarding the occurrence of a detected event.

Additional benefits and advantages of the concepts of the disclosure will be described in detail with reference to the accompanying drawings, or otherwise appreciated by those of ordinary skill in the relevant art upon a review of the present disclosure.

### BRIEF DESCRIPTION OF THE SEVERAL VIEWS OF THE DRAWINGS

The present disclosure will be more fully understood by reference to the following figures, which are for illustrative purposes only. These non-limiting and non-exhaustive embodiments are described with reference to the following drawings, wherein like labels refer to like parts throughout the various views unless otherwise specified. The sizes and relative positions of elements in the drawings are not necessarily drawn to scale in some figures. For example, the shapes of various elements are selected, enlarged, and positioned to improve drawing legibility. In other figures, the sizes and relative positions of elements in the drawings are exactly to scale. The particular shapes of the elements as drawn may have been selected for ease of recognition in the drawings. The figures do not describe every aspect of the teachings disclosed herein and do not limit the scope of the claims.

FIG. 1A is an isometric view of an embodiment of a detection system including a lighting device, a receiver coupled to the lighting device, and a detection unit removably insertable in the receiver and interfacing with the lighting device according to the present disclosure.

FIG. 1B is an isometric view of the detection system of FIG. 1A mounted on headgear.

FIG. 1C is an isometric view of the lighting device of FIG. 1A without the receiver and the detection unit.

FIG. 2 is an isometric view of the receiver of FIG. 1A in a form factor of an adapter.

FIG. 3 is a cross-sectional view of the adapter of FIG. 2 along line A-A in FIG. 2.

FIG. 4 is an isometric view of an embodiment of a detection system including a receiver coupleable to headgear and a detector unit in a form factor of a functional detector cartridge removably received in the receiver according to the present disclosure.

FIG. 5 is an isometric view of the receiver of FIG. 5 in a form factor of a clip.

FIG. 6A and FIG. 6B are isometric views of an embodiment of a detector unit in a form factor of a functional detector cartridge.

FIG. 7 is a block diagram of a controller suitable for executing an embodiment of a detection system that performs at least some techniques described in the present disclosure, as well as various devices and/or computing systems connected thereto.

FIG. 8A is a schematic diagram providing an overview of network and communication operations according to an embodiment of a detection system of the present disclosure.

FIG. 8B is a block diagram of a computing system suitable for executing an embodiment of a system that performs at least some techniques described in the present disclosure, as well as various devices and/or computing systems connected thereto.

FIG. 9 is a graphical representation illustrating at least some techniques of a threshold adaptive mode of an embodiment of a detection system of the present disclosure.

### DETAILED DESCRIPTION

Persons of ordinary skill in the relevant art will understand that the present disclosure is illustrative only and not in any way limiting. Other embodiments of the presently disclosed systems and methods readily suggest themselves to such skilled persons having the assistance of this disclosure.

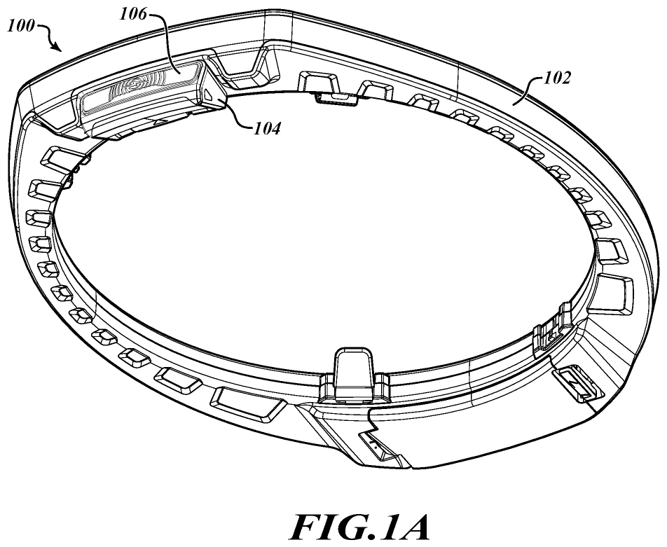

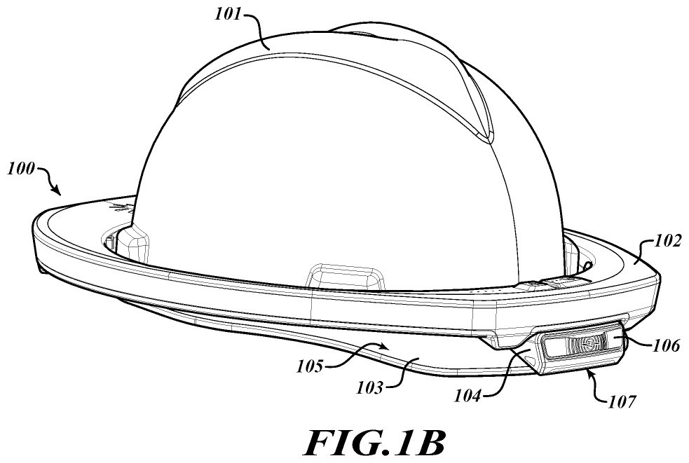

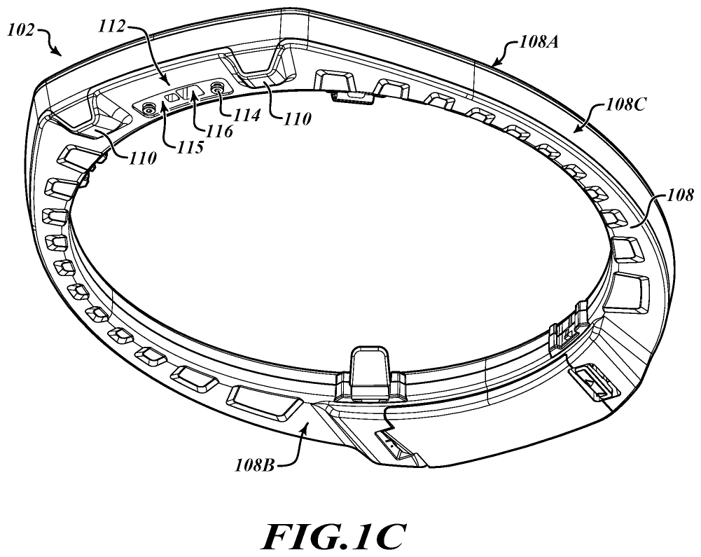

Each of the features and teachings disclosed herein can be utilized separately or in conjunction with other features and teachings to provide detection devices, systems, and methods. Representative examples utilizing many of these additional features and teachings, both separately and in combination, are described in further detail with reference to attached FIGS. 1-9. This detailed description is merely intended to teach a person of skill in the art further details for practicing aspects of the present teachings and is not intended to limit the scope of the claims. Therefore, combinations of features disclosed in the detailed description may not be necessary to practice the teachings in the broadest sense, and are instead taught merely to describe particularly representative examples of the present teachings.

Moreover, the various features of the representative examples and the dependent claims may be combined in ways that are not specifically and explicitly enumerated in order to provide additional useful embodiments of the present teachings. It is also expressly noted that all value ranges or indications of groups of entities disclose every possible intermediate value or intermediate entity for the purpose of original disclosure, as well as for the purpose of restricting the claimed subject matter. It is also expressly noted that the dimensions and the shapes of the components shown in the figures are designed to help understand how the present teachings are practiced, but are not intended to limit the dimensions and the shapes shown in the examples in some embodiments. In some embodiments, the dimensions and the shapes of the components shown in the figures are exactly to scale and intended to limit the dimensions and the shapes of the components.

Detection systems, devices, and methods are described herein for identifying risks for workers and other users in an environment. In addition, techniques are described for configuring and facilitating alert notifications regarding detected events, and may further provide various analyses of tracked information related to such users, alerts, and notifications. Some or all of the techniques described herein may be performed by automated operations of an embodiment of the detection devices, systems, and methods, as discussed in greater detail below, which devices, systems, and methods may include one or more signaling devices in the nature of a form factor and/or a wearable lighting device.

As used herein, the term “user” may refer to any human operator of a device or system described in the present disclosure, while the term “worker” may refer to any user associated with a particular signaling device of such system. The term “worksite” may refer to any geographical location associated with one or more of such workers, and may include recreational as well as industrial and professional locations. The term “selecting,” when used herein in relation to one or more elements of a graphical user interface or other electronic display, may include various user actions taken with respect to various input control devices depending on the client computing device used to interact with the display, such as one or more clicks using a mouse or other pointing device, one or more tapping interactions using a touch screen of a client device, etc. In addition, such selecting may additionally comprise interactions with various physical actuators capable of generating electrical or electronic signals as a result of such interactions. A nonexclusive list of examples of such actuators include electronic, mechanical or electromechanical implementations of keys, buttons, pressure plates, paddles, pedals, wheels, triggers, slides, touchpads, or other touch- or motion-sensitive element, and may be digital or analog in nature. Also as used herein, unless specifically disclaimed any “alert” or “warning” may incorporate visual, auditory, haptic, or other information conveyed to a user.

In certain embodiments, multiple workers proximate to a geographical worksite are each associated with a detection system that includes at least a detector unit. The detector unit is in one of multiple form factors, including but not limited to a functional detector cartridge and has at least one status indicator, such as, for example a lighting element, lighting array including a plurality of LED or other lighting elements, a buzzer, and/or a speaker. The detector unit may include a power source in electronic communication with the at least one status indicator. The lighting array may comprise various physical configurations of the individual lighting elements, and in at least some embodiments such individual lighting elements may be position- and hue-addressable, such as to control the color and state of each element independently of or in conjunction with the other elements of the lighting array. In addition, the detector unit may include a controller having at least one processor or microcontroller, one or more memories storing various configuration information and other computer-readable instructions (such as in upgradable firmware or other manner), and various communications devices and interfaces for communicating with other devices and additional components of a broader detection system or worksite safety (“WSS”) system. For example, a detector unit may include one or more BLE (“Bluetooth low energy”) and additional component chipsets for transmitting and receiving identification, location or proximity, alert, and status information, and/or other wireless communications components for such functions; in various embodiments, such chipsets may include transceivers, sensor chipsets, and other component chipsets.

In certain embodiments, a detector unit may include an accelerometer, such as to detect sudden acceleration or impact that might indicate possible trauma or injury to the worker wearing or carrying the detector unit, as well as one or more manual controls (e.g., buttons, touch controls, or other physical actuators) in order to activate various functions of the system, such as power and manual selection of security and configuration functionality of the detector unit, as described in greater detail elsewhere herein. In addition, a detector unit may further include various other components, such as audio speakers for conveying audible alerts; vibration-enabled components for conveying haptic alerts; or other components for sensing or conveying particular information to the wearer or carrier of the detector unit.

In at least some embodiments, the detector unit is associated with a lighting device, such as a wearable or carryable lighting device in some non-limiting examples. For example, the communication components and functionality of the detector unit above may enable wireless interaction and communication between the detector unit and the lighting device. The detector unit may also be connected to the lighting device by a wired connection or otherwise in communication with the lighting element through physical electrical connectors. The lighting device may include, for example, a standard headlamp wearable on headgear as well as a signaling device in at least a partially ring-shaped form factor (occasionally referred to herein as simply a ring signaling device), among other lighting devices. The lighting device may have a power source that is configured to provide power from the lighting device to the detector unit to increase the overall operational life of the detector unit during use and between charges. In addition, the controller of the detector unit may send instructions, signals, or other data to change at least one characteristic of the lighting device in response to a detected event, such as, for example, a color or hue of a lighting element or lighting array of the wearable lighting device. The lighting device may send instructions, signals, or other data to the detector unit as well.

A ring signaling device as described herein may include certain aspects that are similar to the components of the detector unit. For example, the ring signaling device may include a power source and a lighting array including a plurality of LED or other lighting elements. The lighting array may comprise various physical configurations of the individual lighting elements, and in at least some embodiments such individual lighting elements may be position- and hue-addressable, such as to control the color and state of each element independently of or in conjunction with the other elements of the lighting array. In addition, a ring signaling device may include a controller, microcontroller or other processor, one or more memories storing various configuration information (such as in upgradable firmware or other manner), a GPS interface and chipset for determining a geographical location of the ring signaling device, and various communications interfaces for communicating with other ring signaling devices and additional components of a broader safety system. For example, a ring signaling device may include one or more BLE (“Bluetooth low energy”) and additional component chipsets for transmitting and receiving identification, location, and status information, and/or other wireless communications components for such functions; in various embodiments, such chipsets may include transceivers, sensor chipsets, and other component chipsets. The ring signaling device may be provided in the form of or include features the same or similar to the Halo™ or Halo SL™ light-emitting products offered by Illumagear Inc., the applicant of the present application.

In certain embodiments, a ring signaling device may include an accelerometer, such as to detect sudden acceleration or impact that might indicate possible trauma or injury to the worker wearing the ring signaling device, as well as one or more manual controls (e.g., buttons, touch controls, or other physical actuators) in order to activate various functions of the ring signaling device such as power and manual selection of particular lighting schemes or modes. In further embodiments, the ring signaling device may omit the accelerometer where the accelerometer is instead present in the detector unit and vice versa. In addition, a ring signaling device may further include various other components, such as one or more biometric sensors, such as to detect biometric information regarding a user of the ring signaling device (e.g., a pulse rate sensor, carbon monoxide or carbon dioxide sensor, etc.) and provide related alerts; audio speakers for conveying audible alerts; vibration-enabled components for conveying haptic alerts; or other components for sensing or conveying particular information to the wearer of the ring signaling device.

Each ring signaling device may have one of multiple form factors. For example, a ring signaling device may substantially comprise a ring form factor that is mountable or otherwise removably interfaced with a standardized “hard hat” or other safety-oriented headgear, similar to, for example, the light-emitting devices shown and described in U.S. Pat. No. 8,529,082, which is assigned to the present applicant, Illumagear Inc., and the content of which is incorporated herein by reference in its entirety. In such use, the particular elements of the ring signaling device's lighting array may be arranged such that the lighting array forms a band around the head of the associated user that is n-elements wide. In other embodiments, the ring signaling device may be integrated into such rigid headgear directly (i.e., not removable from the hard hat). As used elsewhere herein, a ring signaling device may be assumed to comprise the removable ring form factor described above unless context indicates otherwise.

In still other embodiments, the detector unit may be in communication with additional signaling devices, form factors, and detection devices in a broader WSS system or safety network. A signaling device may comprise a vest, a mesh grid, wristbands, attachments for independently worn items such as clothing and jewelry, or other wearable form factor. In other instances, the signaling device may comprise a non-wearable form factor, such as an appropriately configured road flare or traffic cone, which may be controlled to illuminate in accordance with the various functionalities described herein. Additional detection devices may include a carbon monoxide sensor, a gas sensor, a smoke sensor, and other like sensors indicating hazardous work conditions in various form factors.

In operation, a plurality of detector units proximate to a particular worksite may maintain communication with each other and with one or more gateway base stations or other networking systems associated with the worksite. In certain embodiments, the detector units may provide information or signals pertaining to detected events, such as for example, a detected electrical field or a fall event to one another (such as via a mesh network), or directly to a site management server. In certain scenarios, implementing multiple gateway base stations across a single large worksite may allow the systems disclosed herein to extend the communication range for connected detector units while limiting the power consumption or wireless communications hardware required for such units to maintain a connection with the system. In addition, in various implementations, communications components having a longer range may be incorporated into the detector units such that the connectivity of the detector units is extended without incorporating a gateway base station into the network.

In certain embodiments, a WSS system in communication with the detector units may provide alerts in a manner other than changing at least one characteristic of the detector units or the at least one status indicator thereof. For example, the WSS system may initiate one or more notifications to users of the WSS system via an application associated with the WSS system and executing on one or more devices associated with such users; via haptic or audible alert; via electronic text messaging, electronic mail, or other textual communication; or other appropriate messaging. As one particular example, if an impact alert is received from a detector unit, one alert may be provided by causing the particular detector unit to emit a predefined lighting scheme or audible signal or warning, while another alert may be simultaneously provided to one or more computing devices associated with additional users of the WSS system, such as a safety manager, worksite supervisor, medical personnel, etc.

Various operations of the WSS system may be configured using a site management computing system communicatively coupled to one or more gateway base stations that are associated with a worksite. In certain embodiments and scenarios, a site management server may be associated with multiple worksites, so as to track and configure devices across those multiple worksites, using consistent configuration parameters across such worksites or using multiple worksite-specific configurations for the corresponding devices. The WSS system may provide a user of the site management server with a visual display indicating the status of all connected devices and/or gateway base stations. In addition, the WSS system may provide a user of the site management server with additional functionality, including but not limited to: configuring subgroups of workers across unique communication channels associated with subgroups of detector units; configuring events to be associated with particular notifications and/or detector unit status indicator schemes; defining criteria for identification of and/or or use of the detector units by one or more workers and corresponding detector units; etc.

In some embodiments, certain operations of the WSS system and one or more detector units may be configured using a worker client device executing an application associated with and/or provided by the WSS system. In addition, a worker associated with a detector unit may also configure particular aspects of the detector unit using the worker client device, such as by establishing a link (e.g., a Bluetooth connection) between the client device and the detector unit. For example, a worker may select to receive particular audible and/or visual alerts via the associated client device and configure a detection sensitivity of certain aspects of the detector unit, in addition to any detection unit status indicator scheme associated with such alerts. In yet further examples, the detector units may have a manual input corresponding to security functionality before a user can configure settings of the detector unit through the worker client device.

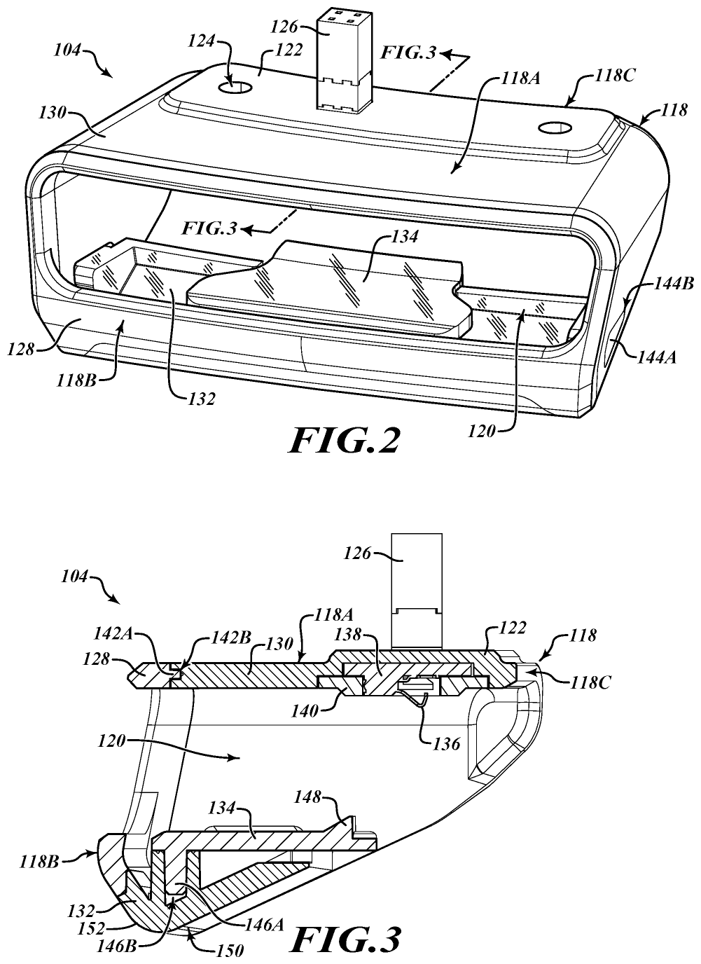

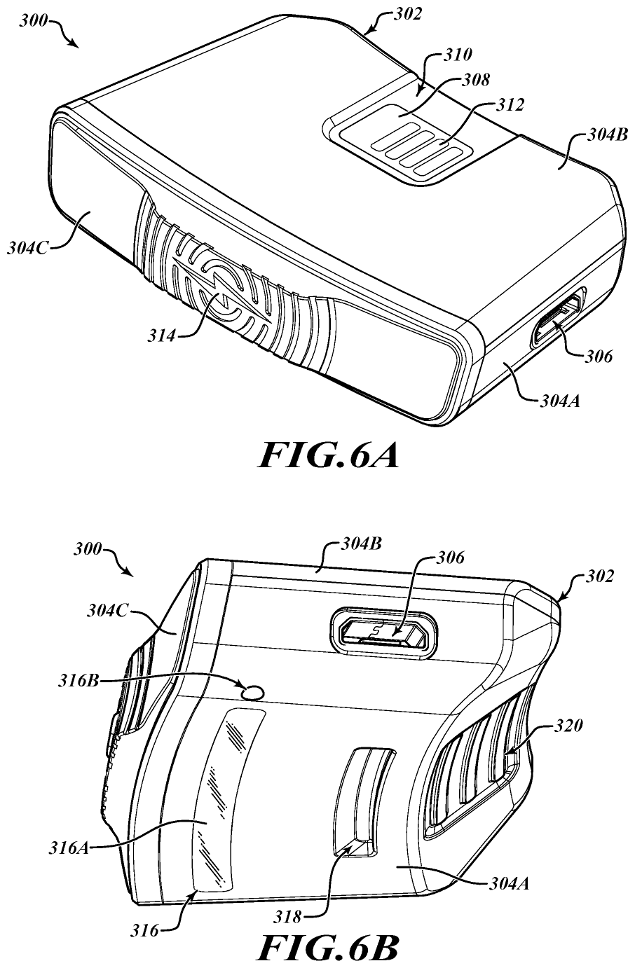

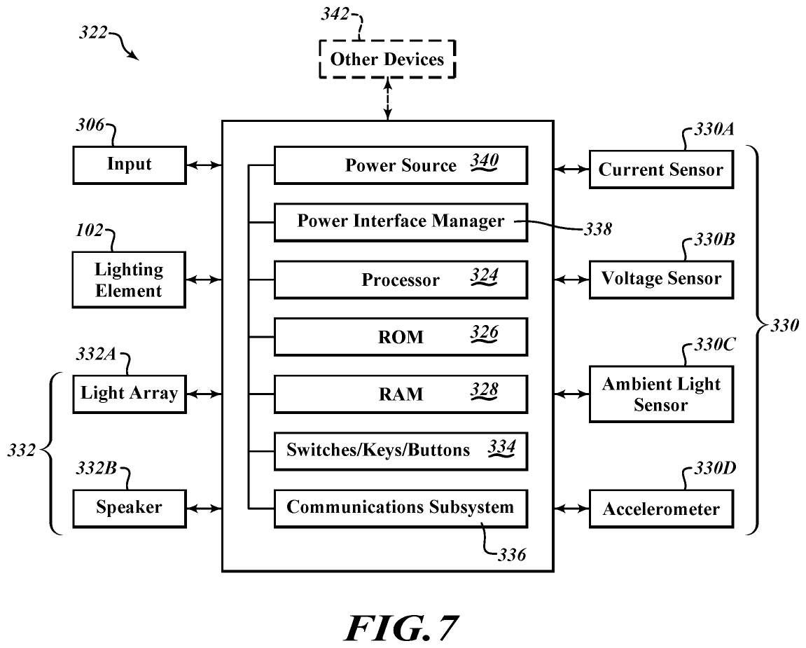

FIG. 1A, FIG. 1B, and FIG. 1C are isometric views of a detection system 100, which may be a combined detection and signaling system including at least a detection component and a signaling component in the same or different form factors, as explained further below. Beginning with FIG. 1A, the detection system 100 generally includes at least a lighting device 102, a receiver 104 removably coupled to, or removably coupleable to, the lighting device 102, and a detector unit 106 removably inserted in the receiver 104. The receiver 104 will be described in additional detail with reference to at least FIG. 2 and FIG. 3 and the detector unit 106 will be described in additional detail with reference to at least FIG. 6A, FIG. 6B, and FIG. 7 of the present disclosure.

Briefly, and to provide additional context regarding the concepts of the disclosure, the receiver 104 is removably coupleable to the lighting device 102 and structured to removably receive the detector unit 106. Or, in other words, the detector unit 106 is removably insertable into the receiver 104 to couple the detector unit 106 to the lighting device 102 via the receiver 104. The receiver 104 enables interaction between the lighting device 102 and the detector unit 106, such as, for example, to provide power from the lighting device 102 to the detector unit 106, to enable communication of signals, computer-readable instructions or other data from the lighting device 102 to the detector unit 106, and/or to enable communication of signals, computer-readable instructions, or other data from the detector unit 106 to the lighting device 102.

The detector unit 106 is configured to detect at least whether a voltage is active in an environment in a vicinity of the user, whether an electrical current is active in an environment in the vicinity of the user, and whether a fall event associated with the user of the detector unit 106 has occurred. The detector unit 106 may have at least one status indicator along with a controller including a memory and at least one processor executing instructions stored in the memory for changing at least one characteristic of the status indicator in response to a detected event, as explained in more detail with reference to at least FIG. 7. The detector unit 106 is further configured to detect such events and issue warnings or alerts via the status indicator in real time and on a continuous or substantially continuous basis, as further explained below, to ensure that alerts or warnings of changing conditions are provided to a worker in real time or near real time. In some embodiments, the detector unit 106 may transmit one or more signals, computer-readable instructions, or other data to the lighting device 102 to likewise change at least one characteristic of the lighting element in response to a detected event.

The lighting device 102 may be provided in one of a number of different form factors. Although the lighting device 102 is illustrated as a ring signaling device according to at least one embodiment shown in FIG. 1A, FIG. 1B, and FIG. 1C, the lighting device 102 may also be another wearable or carryable lighting device, such as for example, a headlamp, a vest with a light array, gloves with a light array, a flashlight, and other like devices, including in particular other like devices for personal illumination or illumination of an immediate area around a worker or other user at a worksite. The lighting device 102 may generally include the features described above, including a lighting array with individual lighting elements that are at least color- and hue-addressable. The lighting device 102 may also include GPS functionality for providing location and identification information or data for users of the detection system 100. The lighting device 102 may generally be similar to the lighting elements, devices, systems, and methods shown and described in U.S. Pat. No. 8,529,082 filed on Feb. 27, 2013 and entitled “Light-Emitting Systems for Headgear” and U.S. patent application Ser. No. 17/282,724 filed on Apr. 2, 2021 and entitled “Light-Emitting Systems,” both of which are assigned to the present applicant, Illumagear Inc., and the contents of the both of which are incorporated herein by reference in their entirety. The lighting device 102 may be provided in the form of or include features the same or similar to the Halo™ or Halo SL™ light-emitting products offered by Illumagear Inc., the applicant of the present application.

The lighting device 102 is in communication with the detector unit 106 and may interface with the detector unit 106, or otherwise cooperate with the detector unit 106, to perform certain functionality and techniques of the detection system 100. In an embodiment, each of the lighting device 102 and the detector unit 106 are individual detection and signaling devices, but that may also cooperate to form a larger detection and signaling device as part of the detection system 100 to expand the overall functionality of the system 100. For example, the lighting device 102 may have a power source, such as a rechargeable or replaceable battery, that may be larger than a power source of the detection unit 106 due to the difference in form factor between the two devices. As a result, the lighting device 102 may provide power to the detector unit 106 to increase the operational lifecycle of the detector unit 106 between charges or between battery replacements. Such an arrangement reduces the likelihood that the detector unit 106 will become inoperative and ineffective at detecting certain events during routine use due to a dead battery.

Further, the detector unit 106 may detect one of the events above and send a signal, computer-readable instructions, and/or other data to the lighting device 102 that cause the lighting device 102 to display a selected alert or warning scheme associated with the event detected by the detector unit 106. In one non-limiting example, the detector unit 106 may detect that a user associated with the system 100 has experienced a fall event, in which case, the detector unit 106 instructs the lighting element to display bright red lights, either static or a in a dynamic and/or animated scheme, indicative of a serious injury or a need for immediate assistance. Alternatively, if the detector unit 106 detects an active voltage an/or current in a vicinity of the user of the system 100, the detector 106 may instruct the lighting device 102 to display a different colored light, such as yellow, to indicate to the individual worker as well as other workers and users in the area of the indicated risk or hazard. Yet further, the detection system 100 may be in communication with other like detection systems associated with different workers or other users who are proximate to each other (i.e., within a predefined threshold distance from one another according to the GPS functionality of the lighting device 102 and/or the detector unit 106) such that other nearby detection systems 100 will likewise execute a selected warning or alert scheme if one detector unit 106 in the area detects a certain event.

In some embodiments, the lighting device 102 may likewise send signals, computer-readable instructions, or other data to the detector unit 106. The lighting device 102 may have additional functionality not found in the detector unit 106, such as at least GPS (i.e., via a GPS chipset), location, and identification functionality. Accordingly, the lighting device 102 may communicate instructions to the detection unit 106 according to the location of the worker or other user associated with the system 100. In a non-limiting example, a WSS system in communication with the lighting device 102 may define certain high risk areas for electrical shock in a larger project. For example, if one building of a multi-building worksite or one floor of a multi-floor building has active voltage and electrical currents, while another building or floor does not, the lighting device 102 may determine when the user associated with the system 100 is within a selected predefined distance from the active electrical area and send instructions to the detector unit 106. The instructions may include, for example, switching the detector unit 106 from a low-power mode with more time between detection cycles to a normal-operation mode and/or a high-alert mode with less time between detection cycles. The instructions may also include adjustment of a sensitivity of the detector unit 106, such as with respect to threshold values before the detector unit 106 issues a warning (i.e., a warning may be issued only for a change or increase in the active voltage and current as opposed to any active voltage or current when the worker is further from the active electrical area or “high risk” area). In yet further examples, the lighting device 102 may receive information from a broader WSS system and convey it to the detector unit 106 to cause the detector unit 106 to issue a warning. For example, if the WSS system sends an alert to the lighting device 102 of a particular risk in an area, or an event detected by another lighting device 102 and/or detector unit 106 at the worksite, the lighting device 102 may transmit the information to the detector unit 106 and cause the detector unit 106 to issue a particular warning scheme, which may be a haptic, audible, and/or visual warning or alert via the status indicator of the detector unit 106.

Thus, in view of the above, the lighting device 102 and the detector unit 106 may cooperate to expand the functionality of the system 100 while also enhancing the signaling capabilities of the system 100 (i.e., the lighting device 102 and the detector unit 106 interface to simultaneously issue alerts and warnings, with the lighting device 102 potentially enhancing the signaling scheme in a larger form factor). In some embodiments, the lighting device 102 and the detector unit 106 operate independently of each other without departing from the scope of the disclosure and otherwise impacting performance of the system 100. For example, the detector unit 106 may be utilized in conjunction with a conventional headlamp without the capabilities of a ring signaling device and still achieve the benefits and advantages described herein. However, the interface between the lighting device 102 and the detector unit 106 according to some embodiments may further reduce certain risks to workers and other users and provide a more comprehensive detection system 100. As will be described in more detail below, the receiver 104 may have a form factor that enables interfacing between the lighting device 102 and the detector unit 106 via direct or “wired” electrical connection, or the lighting device 102 and the detector unit 106 may in communication wirelessly according to various communication protocols.

The detection system 100 may further include other detector devices or sensors associated with the system 100, either coupleable to the lighting device 102 or otherwise wearable on other protective equipment or gear of a worker or other user and/or carryable by a worker or other user associated with the detection system 100. For example, a carbon monoxide or noxious and/or toxic gas sensor may be integrated into the system 100, either as part of the detector unit 106, or in a separate form factor coupleable to the lighting device 102. The gas sensor may also be in a form factor separately wearable or carryable by the user. As described above, the gas sensor may likewise be in communication with components of the system 100 and may include standalone signaling capabilities or may interface with the components of the system 100, such as the lighting device 102, to enhance the signaling capabilities in response to a detected event by the gas sensor. Other detector devices are contemplated herein, including but not limited to oxygen detectors, smoke detectors, heat detectors, biometric detectors, and other like devices that may likewise operate independently as a detection and signaling device, or may interface with the system 100.

FIG. 1B is an isometric view of the detection system 100 mounted on headgear 101. The headgear 101 may include a brim 103 with a top surface 105 generally facing away from a head of the worker or other user. In an embodiment, the detection system 100 at least partially rests, or is otherwise disposed on the top surface 105 of the brim 103. In particular, the receiver 104 of the detection system 100 interfaces with the lighting device 102 above the brim 103, and/or above the top surface 105 of the brim 103. However, the detector unit 106 is arranged in the detection system 100 such that a signal emission portion 107 of the detector unit 106 and/or the receiver 104 are in a line of sight of a worker or other user wearing the detection system 100 and the headgear 101. As further explained below, the signal emission portion 107 of the detector unit 106 and/or the receiver 104 are configured to emit one or more signals, warnings, or other alerts, which may include particular lighting, sound, and/or haptic signaling schemes. The detector unit 106 and/or the receiver 104 are configured to emit at least such visual and/or lighting signals in the line of sight of the worker or other user despite the system 100 being mounted above the brim 103 of the headgear 101. Accordingly, the location of the detection system 100 above the brim 103 may generally place the detection system 100 out of the line of sight of the worker or other user during normal operation, while also enabling signals or other alerts to be clearly provided in the line of sight of the worker or other user in response to a detected event in order to alert such worker or other user. Such an arrangement provides an advantageous solution because the worker or other user is not distracted by the detection system 100 being in their line of sight during use, but are also appropriately alerted or otherwise signaled in response to an occurrence of a detected event.

Turning to FIG. 1C, the lighting device 102 is illustrated without the receiver 104 and the detector unit 106 to provide additional detail regarding the lighting device 102. The lighting device 102 includes a form factor 108 with a first surface 108A and a second surface 108B opposite to the first surface 108A with a third surface 108C between the first and second surfaces 108A, 108B. In an embodiment, the form factor 108 is generally ring-shaped and configured to be worn on headgear, such as a hard-hat, among other safety devices. The form factor 108 may also be permanently incorporated into headgear. The first surface 108A is a top surface of the form factor 108 and the second surface 108B is a bottom surface in some embodiments with the third surface 108C being a side surface or a sidewall surface 108C extending between the first and second surfaces 108A, 108B.

The lighting device 102 may include a lighting array or at least one individual lighting element of the type described herein on or associated with the third surface 108C such that the lighting device 102 is configured to radiate light outward in at least a partial, or a complete, circle around the worker. The form factor 108 also includes opposing projections 110 on the second surface 108B proximate a front of the lighting device 102 that define a receiving space 112. The receiver 104 is configured to be removably received within the receiving space 112. In an embodiment, the projections 112 may be located on any of the surfaces 108A, 108B, 108C and on any side of the lighting device 102 (i.e., front, back, left, and/or right, and anywhere in between). In addition, an outward-facing surface of the projections may likewise include at least one lighting element or lighting array, with the projections 110 generally tapering or otherwise gradually or continuously decreasing in height from the outward-facing surface to an interface between the projections 110 and the form factor 108.

The form factor 108 further includes, at the receiving space 112, one or more mounting fasteners 114 for securing the receiver 104 to the form factor 108. However, other securing devices and/or mechanisms are contemplated herein as viable replacements for the one or more mounting fasteners 114, including but not limited to latches, friction fit or snap fit components or assemblies, buckles, hook and loop fasteners, and other like devices. The mounting fasteners 114 may include two fasteners 114, or only one fastener 114 or more than two fasteners 114, such as three, four, five, or more fasteners 114. The fasteners 114 interface with a corresponding structure on the receiver 104 described in more detail with reference to FIG. 2 to couple the receiver 104 to the lighting device 102. Further, an electrical connector 116 is positioned in the receiving space 112 for interfacing with a corresponding structure on the receiver 104. In an embodiment, the electrical connector 116 is a female electrical connector or socket for receiving an electrical connector of the receiver 104. In some embodiments, the electrical connector 116 may also receive a corresponding connector directly on the detector unit 106.

The electrical connector 116 may be any one of a number of different form factors and/or connectors and/or sockets for establishing an electrical connection or otherwise enabling communication or interfacing between devices, electrical or otherwise. In an embodiment, the electrical connector 116 enables interfacing or other communication between the lighting device 102 and/or the receiver 104 and/or the detector unit 106. The electrical connector 116 may be located between the mounting fasteners 114 in the receiving space 112 and may be recessed with respect to a surface of the receiving space 112 along with the mounting fasteners 112. In more detail, the mounting fasteners 114 and the elector connector 116 are positioned in a recess 115 of the lighting device 102 with the recess 115 located in the receiving space 112. The recess 115 may be positioned closer to an inner surface of the lighting device 102 (i.e., a surface configured to face the worker or other user and/or headgear during operation) than an outer surface (i.e., a surface configured to face away from the worker or other user and/or headgear during operation) of the lighting device 102 in an embodiment, although the same is not necessarily required. As described below with reference to FIG. 2, the recess 115 may interface with a platform of the receiver 104 to prevent dust, water, and other contaminants from reaching the electrical connector 106 when the receiver 104 is coupled to the lighting fasteners 102. In other words, the recess 115 and corresponding platform of the receiver 104 increase the length of the path of travel, and also the elevation of the path of travel, for potential contaminants to reach the electrical connector 116.

FIG. 2 is an isometric view of the receiver 104. As will be described in more detail below, the receiver 104 according the present disclosure may be provided in the nature of a number of different form factors capable of being attached to headgear, safety gear, or any other item wearable or carryable by a worker or other user. In FIG. 2, the receiver 104 is provided in the form factor of an adapter configured to interface with both the lighting device 102 and the detector unit 106 (FIG. 1A) to enable communication between the lighting device 102 and the detector unit 106 of the type described herein via the adapter. For clarity between the embodiments of the disclosure, the receiver 104 will be referred to as an adapter 104 with reference to FIG. 2 and FIG. 3. However, it is to be appreciated that “receiver” is to be construed broadly to include a variety of form factors, as above, with “adapter” being a subset of a “receiver” capable of enabling interfacing, communication, and/or interaction between two components, including but not limited to the lighting device 102 and the detector unit 106 (FIG. 1A). Thus, the receiver 104 according to the present disclosure may have the features of the adapter 104 described below with reference to FIG. 2 and FIG. 3 as well as other form factors of the receiver 104 described herein.

The adapter 104 includes a form factor 118 having a first surface 118A, a second surface 118B, and a third surface 118C. In an embodiment, the first surface 118A is a top surface, the second surface 118B is a front surface, and the third surface 118C is a rear surface. As a result, the second and third surfaces 118B, 118C are opposite each other. The form factor 118 generally defines a slot 120, which may also be referred to herein as a detector unit slot 120. The detector unit slot 120 extends through the form factor 118 from the second surface 118B to the third surface 118C and generally has a size and a shape corresponding to the detector unit 106 (FIG. 1A). The detector unit 106 is removably insertable into the detector unit slot 120 of the adapter 104, as best shown in FIG. 1A.

In some embodiments, the adapter 104 includes a platform 122 extending from the first surface 118A. The platform 122 may cover more or less than a majority of the first surface 118A and may be positioned closer to the third surface 118C than the second surface 118B, or adjacent the third surface 118C in non-limiting examples. As noted above, the platform 122 is configured to interface with the recess 115 of the receiving space 112 of the lighting device 102, or more specifically, the platform 112 is removably insertable in the recess 115 of the lighting device 102 to prevent contaminants from interacting with the electrical components of the system 100. At least one hole 124 extends through the platform 122 and the first surface 118A of the form factor 118 such that the at least one hole 124 is in communication with the slot 120. The at least one hole 124 is configured to receive the one or more mounting fasteners 114 (FIG. 1C) to couple the adapter 104 to the lighting device 102 (FIG. 1A).

Accordingly, the number of at least one holes 124 may correspond to the number of mounting fasteners 114 in an embodiment. An electrical connector 126 is coupled to the form factor 118, and specifically, is coupled to and extends from the platform 122. The electrical connector 126 may be a male electrical connector of a selected type configured to interface with the electrical connector 116 of the lighting device 102 (FIG. 1C). In an embodiment, the electrical connector 126 is a post-type electrical connector with one or more pins or electrical contacts electrically coupled to conductors (e.g., wires) running through a post or body of the connector 126. In operation, the electrical connector 126 of the adapter 104 is removably insertable in the electrical connector 116 of the lighting device 102 (FIG. 1C) such that when the detector unit 106 is received in the slot 120, the detector unit 106 interfaces with the lighting device 102 via the adapter 104 and the electrical connectors 116, 126. In an embodiment, the electrical connector 126 is offset from a center of the platform 122 and is positioned closer to the one of the at least one holes 124 than others of the at least one holes 124, although the position and arrangement of the electrical connector 126 can be selected according to design factors.

The form factor 118 includes a face plate 128 coupled to a case 130 with the case 130 defining the first surface 118A and the third surface 118B and the face plate 128 defining the second surface 118B. A window 132 is coupled to the face plate 128 and the case 130 with the window 132 configured to receive a latching device 134. In an embodiment, the window 132 and the latching device 134 are transparent, or at least have a translucence that allows a majority of light to pass through the window 132 and the latching device 134. In operation, and as explained further below, the detector unit 106 emits light generally downward toward the worker or other user that passes through the window 132 and the latching device 134 to illuminate an area in front of the worker or other user and signal occurrence of a detected event to the worker or other user. In one or more embodiments, the face plate 128 and the case 130 are opaque, or at least have a translucence that blocks a majority of light to prevent light from the detector unit 106 from escaping through the face plate 128 and the case 130. The face plate 128 and the case 130 may be plastic with a light-blocking additive to reduce the translucence of the face plate 128 and the case 130. The face plate 128 and the case 130 may also be transparent in embodiments where the detector unit 106 is (FIG. 1A) is further configured to emit light toward a front or above a worker or other user as an additional signaling technique.

FIG. 3 is a cross-sectional view of the adapter 104 along line A-A in FIG. 2. As shown in FIG. 2, the adapter 104 includes at least one electrical contact 136 in communication with the electrical connector 126. In one non-limiting example, the electrical contact 136 is one of a series of pins mounted on, and extending from, a circuit board 138 or printed circuit board 138. The electrical connector 126 is likewise mounted on the circuit board 138 such that the electrical connector 126 is in electrical communication with the electrical contact 136 for transmitting power, one or more signals, instructions, and/or other data from an electrical system of the lighting device 102 (FIG. 1A) to the electrical contact 136. The electrical connector 126 and the electrical contact 136 are mounted on opposite sides of the circuit board 138, such as the connector 126 being a top side of the board and the contact 136 being on the bottom side of the board 138. In an embodiment, the circuit board 138 may instead be a metal plate or the electrical contact 136 may be in communication with the electrical connector 126 via a wired connection. The circuit board 138 is coupled to the case 130 by a cover plate 140, which also prevents contaminants from reaching the circuit board 138.

FIG. 3 also provides additional detail regarding the connection between various structures of the form factor 118. More specifically, the face plate 128 may be coupled to the case 130 with a tongue and groove connection, meaning that the face plate 128 has a tongue 142A received in a corresponding shaped groove 142B in the case 130. In an embodiment, the face plate 128 is attached to the case 130 with assistance from adhesive or by welding, such as by ultrasonic welding, to fuse the parts together. The window 132 is coupled to the case 130 by a snap fit connection best shown in FIG. 2. In particular the window 132 has protrusions 144A on opposite sides of the window 132 that snap into place in openings 142B on opposite sides of the case 130. Returning to FIG. 3, the latching device 134 is coupled to the window 132 and includes an insert 146A received in a channel 146B of the window 132. In an embodiment, the latching device 134 is coupled to the window 132 by adhesive, such as optically clear adhesive, or by welding, such as by ultrasonic welding. The latching device 134 further includes a securing mechanism 148 that interfaces with the detector unit 106 (FIG. 1A) to secure the detector unit 106 in the slot 120. The securing mechanism 148 may have a number of different form factors, including but not limited to a ridge and/or flange shape shown in FIG. 2 and FIG. 3.

A body of the latching device 134 rests on the window 132 and may have at least some elasticity such that the latching device 134 is movable with respect to the window 132. In other words, a worker or other user can manipulate the latching device 134 up and down with respect to the window in the orientation of FIG. 3. Movement of the latching device 134 enables the worker or other user to selectively insert and remove the detector unit 106 (FIG. 1A) in the slot 120 of the adapter 104. Alternatively, the latching device 134 may be rigid and fixed in place, with the worker or other user removably inserting the detector unit 106 (FIG. 1A) in the slot 120 through a force fit (i.e., the worker presses on the detector unit 106 with enough force to secure the detector unit 106 to the latching device 134 and vice versa). The channel 146B of the window 132 may also be referred to herein as a light channel 146B or a light tunnel 146B that receives and transmits light from the detector unit 106 and distributes it throughout a bottom surface 150 of the window 132. As shown in FIG. 3, the bottom surface 150 may have a “V” shape terminating in a vertex 152 for focusing light downward toward the worker or other user.

Thus, the adapter 104 enables the lighting device 102 to interface with the detector unit 106 (FIG. 1A) such that the lighting device 102 and the detector unit 106 can exchange power, one or more signals, instructions or other data, as described throughout the disclosure. In addition, the shape and arrangement of the form factor of the adapter 104 improves the signaling techniques of the system 100, namely by illuminating the window 132 in a line of sight of the worker or other user.

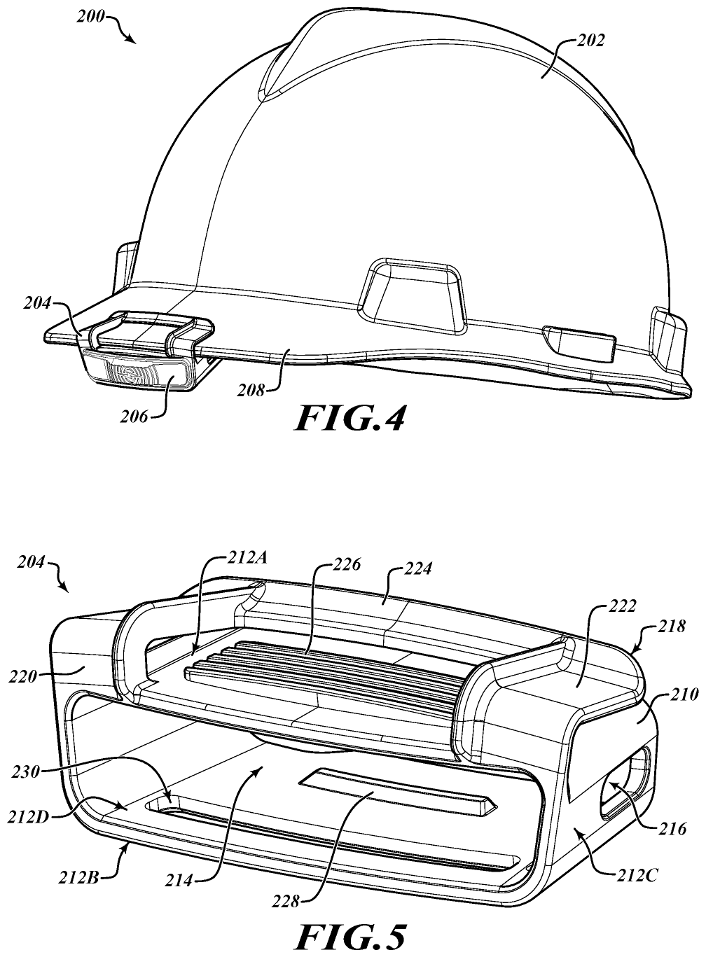

FIG. 4 is an isometric view of an embodiment of a detection system 200. The detection system 200 includes headgear 202 or other safety equipment, a receiver 204 removably coupled to the headgear 202 and a detector unit 206 removably inserted in the receiver 204. The receiver 204 will be described in more detail with reference to FIG. 5. The detector unit 206 may be similar to the detector unit 106 (FIG. 1A) and will be described in more detail with reference to FIG. 6A, FIG. 6B, and FIG. 7. Although FIG. 4 illustrates the receiver 204 coupled to headgear 202, the present disclosure contemplates the receiver 204 coupled to a variety of safety gear or other items that may be worn or carried by a worker or other user. As will be explained further below, the receiver 204 may have a form factor of a clip, and thus the receiver 204 may be coupled to any item worn or carried by a worker or other user than can receive a clip and/or that a clip that may be attached to. Further, the headgear 202 may include a lighting device, such as lighting device 102, or may not include a lighting device 102. Thus, the advantages of the disclosure with respect to the detector unit 206 can be realized without a lighting device. In yet further embodiments, a standard headlamp or other illumination device is associated with the headgear 202 and interfaces with the detector unit 206 to provide at least some, if not all, of the functionality described above with respect to the lighting device 102 (FIG. 1A). For example, in an embodiment, the detector unit 206 may receiver power from the headlamp or other illumination device and/or may communicate with the headlamp or illumination device to change at least one characteristic of the headlamp or other illumination device (i.e., to use the headlamp or other illumination device as a further signaling device).

As shown in FIG. 4, the headgear 202 has a brim 208 with the receiver 204 removably coupleable to the brim 208. The location of the receiver 204 along the brim 208 may be selected by a worker or other user. The receiver 204 is preferably located on the brim 208 to enable the detector unit 206 to provide advantageous signaling techniques to the worker or other user. For example, the detector unit 206 may direct light generally downward as a warning or notification to the worker or other user. Thus, the receiver 204 is preferably located on the brim 208 to position the detector unit 206 in a location where the light emitted by the detector unit 206 is easily visible to the worker or other user and therefore has an advantageous signaling function. When the receiver 204 is located on the brim 208, a majority of the detector unit 206 is covered by the brim 208. Although the detector unit 206 may be waterproof, water resistant, sealed, and/or hermetically sealed in an embodiment, the brim 208 may still assist with preventing large amounts of rainwater or other contaminants from contacting the detector unit 206, which increases the expected operational life of the detector unit 206. However, the receiver 204 may also positioned anywhere on the headgear 202 or attached to other equipment of the worker or other user in some embodiments.

FIG. 5 is an isometric view of the receiver 204. In FIG. 5, the receiver 204 is provided in the form factor of a clip attachable to headgear or other equipment or apparel of a worker or other user. For clarity between embodiments of the disclosure, the receiver 204 will be referred to as a clip 204 in the description of FIG. 5. As with the adapter 104 described with reference to FIG. 2 and FIG. 3, the “clip” should be interpreted as a subset of the broader category of “receiver,” and thus, a receiver according to the disclosure can have the features of the clip 204 described below, as well as others, such as least the features of the adapter 104, or any combination thereof.

The clip 204 has a form factor 210 with a first surface 212A, a second surface 212B, a third surface 212C, and a fourth surface 212D. The first surface 212A may an outer top surface of the form factor 210, the second surface 212B may be an outer bottom surface of the form factor 210 opposite to the first surface 212A, the third surface 212C may be an outer side surface of the form factor 210 between the first and second surfaces 212A, 212B, and the fourth surface 212D may be an interior surface of the form factor 210 and in particular, may be an interior bottom surface in an embodiment. The form factor 210 includes a detector unit slot 214 extending through the form factor 210 and defining a space for receiving the detector unit 206 (FIG. 4). The detector unit 206 (FIG. 4) is removably insertable in the detector unit slot 214 of the form factor 210, as shown at least in FIG. 4 and further described herein. The form factor 210 further includes a connector opening 216 through at least one side surface 212C of the form factor 210. The connector opening 216 may be on the left and/or right side of the form factor 210 in some examples and generally corresponds to inputs of the detector unit 206 (FIG. 4) described further herein.

The form factor 210 of the clip 204 may be a single piece form factor 210 in an embodiment. Further, the clip 204 may have a clamp 218, which may be a C-style clamp, meaning the clamp 218 has arms 220 that extend vertically away from, and generally perpendicular to, the first surface 212A before a body 222 of the clamp 218 turns to extend parallel to, or at an angle towards, the first surface 212A generally in the shape of a “C.” The clamp 218 includes a lip 224 to enable the user to manipulate the clamp 218. In an embodiment, the clamp 218 has at least some elasticity (i.e., the clamp 218 and more particularly, at least the body 222 and the lip 224 move vertically in the orientation shown in FIG. 5) to enable the clamp 218 to engage headgear 202 (FIG. 4) or other equipment of different sizes, shapes, and thicknesses. Further, the arms 220 of the clamp 218 are preferably located on opposite peripheral edges of the form factor 210 to increase a surface area of the clamp 218 in contact with a support, such as headgear 202 (FIG. 4) or other equipment. However, the arms may be positioned at a selected location on the first surface 212A in some embodiments.

The clip 204 has a grip 226 extending from the first surface 212A of the form factor 210 in some embodiments. The grip 226 can have a number of different forms or form factors, including but not limited to one or more ridges arranged in parallel on the first surface 212A. Other form factors of the grip 226 can include one or more dots or bumps, a pad, grip tape, teeth or splines, and other like devices. The grip 226 may also be formed as part of the first surface 212A in some embodiments, such as a portion of the first surface 212A having a greater coefficient of friction than the surrounding form factor 210 to improve engagement between the clip 204 and the headgear 202 or other equipment of a worker or other user. The grip 226 may also be omitted from some embodiments. The grip 226 may be a thermoplastic, elastomer, thermoplastic elastomer, polymer, resin, or other like material that preferably has a higher coefficient of friction than the surrounding material of the form factor 210. In operation, the grip 226 engages the brim 208 of the headgear 202 (FIG. 4), or other equipment of the worker or other user, to improve engagement between the clip 204 and the headgear 202 and reduce the likelihood that the clip 204 will slip or fall from the headgear 202.

In an embodiment, the clip 204 includes a latching device 228 extending from the fourth or interior surface 212D into the detector unit slot 214. The latching device 228 may likewise be provided in a number of different form factors, including but not limited to a ridge or one or more protrusions. A light aperture 230 extends through the form factor 210 from the second surface 212B to the fourth surface 212D and allows light emitted by the detector unit 206 (FIG. 4) to be emitted toward the worker or other user through the clip 204, as described herein. A location of the latching device 228 and the light aperture 230 may be selected or may be omitted in some embodiments according to the form factor of the detector unit 106 (FIG. 4), and may be different than that shown in FIG. 5.

FIG. 6A and FIG. 6B are perspective views of one or more embodiments of a detector unit 300. In particular, FIG. 6A is a top perspective view of the detector unit 300 and FIG. 6B is a bottom perspective view of the detector unit 300. Except as otherwise provided herein, the detector unit 300 may be similar to the detector units 106, 206 and vice versa with the difference in reference numbers merely being for clarity and organization of the disclosure. The detector unit 300 may be provided in a number of different form factors. For example, the detector unit 300 may be integrated into other systems, such as the lighting device 102 or ring signaling devices described herein. In addition, the detector unit 300 may be incorporated into other safety equipment or gear worn or carried by workers or other users. The detector unit 300 may also be in the form of a functional detector cartridge as in FIG. 6A and FIG. 6B. Unless the context dictates otherwise, “detector unit” should be construed broadly to include a number of different form factors with a “functional detector cartridge” being construed as a subset or one group of form factors of a “detector unit” according to the concepts and techniques of the disclosure. In FIG. 6A and FIG. 6B, the detector unit 300 will be referred to as a functional detector cartridge 300 (or simply as a cartridge 300), although it is to be appreciated that embodiments of detector units according to the disclosure can include the features of the functional detector cartridge 300 described herein.

Beginning with FIG. 6A, the functional detector cartridge 300 includes a housing 302 with a lower portion or base 304A, an upper portion or top plate 304B, and a front portion or face plate 304C coupled to each other. In an embodiment, the components of the housing 302 are coupled to each other with adhesive or glue, or may be fused or welded together. An input 306 extends through the housing 302, and particularly through the base 304A of the housing 302 in an embodiment. The input 306 may be a connector and/or socket, including but not limited to a Universal Serial Bus connector and/or a power input, among others. The input 306 is in communication with an electronic system of the cartridge 300 described in more detail with reference to FIG. 7. In operation, a battery of the cartridge 300 may receiver power from an external source to recharge the battery of the cartridge 300, such as a wall outlet and a power cable, via the input 306. In yet further embodiments, the cartridge 300 may interface with one or more external devices via the input 306, such as, for example, a personal computing device or another piece of equipment worn or carried by a worker or other user (i.e., the input 306 can be connected to a headlight, for example, to provide power from the headlight to the cartridge 300 via the input 306) to provide additional functionality.

The cartridge 300 further includes a circuit board 308 or printed circuit board 308 mounted in a recess 310 in the top plate 304B of the housing 302. The circuit board 308 may likewise be in communication with the electrical system of the cartridge 300. The circuit board 308 includes at least one electrical contact 312, such as a contact or metal pad in some embodiments for interfacing with the electrical contact 136 of the adapter 104 shown in FIG. 3. In other words, when the cartridge 300 is inserted into the adapter 104 or other receiver 104, the at least one electrical contact 312 of the cartridge 300 interfaces with the electrical contact 136 of the adapter or receiver 104 to enable communication with the lighting device 102 (FIG. 1A) or other device and the cartridge 300. As noted above, the lighting device 102 or other device may provide power to the cartridge 300 via the contacts 312, or the cartridge 300 may transmit signals, instructions, or other data to the lighting device 102 or other device and receive signals, instructions, or other data from the lighting device 102 or other device. The circuit board 308 is positioned in the recess 310 for interfacing with the raised profile of the cover plate 140 (FIG. 3) of the adapter or receiver 104, which in some cases, may also prevent contaminants and/or moisture from reaching the circuit board 308. In an embodiment, the recess 310 extends from a location proximate a center of the top plate 304B to an outer peripheral edge of the top plate 304B such that the recess 310 is bounded by only three walls as shown in FIG. 6A. The location of the recess 310 may also be selected to be different in some embodiments.

The cartridge 300 may further include an input control device 314 in or on the face plate 304C in the form of a physical actuator or button. As described further with reference to FIG. 7, the input control device 314 may be operable to select an ON/OFF state of the cartridge 300, as well as to enable selection of various operational characteristics or security functionality of the cartridge 300, and/or clear warnings or other signals associated with the cartridge 300, among others. The input control device 314 may be a physical actuator capable of generating electrical or electric signals as a result of manual interaction by a worker or other user with the input control device 314 and may include, but is not limited to, electronic, mechanical or electromechanical implementations of keys, buttons, pressure plates, paddles, pedals, wheels, triggers, slides, touchpads, or other touch- or motion-sensitive element, and may be digital or analog in nature.

Turning to FIG. 6B, the base 304A of the cartridge 300 further includes a signal emission portion 316 that may include at least a transparent strip 316A, or a strip of material 316A having a translucence that allows at least a majority of incident visible light to pass therethrough and an outlet port 316B. The transparent strip 316A is configured to allow light emitted by the electrical system of the cartridge 300 to pass therethrough to notify or warn the worker or other user, as described herein and may also be referred to as a window 316A accordingly. The outlet port 316B may be in communication with a speaker and/or buzzer of the electronic system of the cartridge 300 and is configured to emit sound as an additional type of warning or signal to the worker or other user, either independently of, or in conjunction with, the signal or warning provided by the transparent strip 316A as part of a larger signaling scheme. In an embodiment, the outlet port 316B is in a shape of a cone to amplify sound emitted by the speaker and/or buzzer.

The cartridge 300 also includes a latching device 318 in the base 304A of the cartridge 300 that may be provided in the form of a channel or depression for receiving and interfacing with the latching device 148 of the adapter 104 (FIG. 3) and/or the latching device 228 of the clip 204 (FIG. 5). In some embodiments, the cartridge 300 includes a grip portion 320 at a rear of the cartridge 300 configured to assist the worker or other user with manipulating the cartridge 300. For example, the grip portion 320 may be provided in a similar form to the grip 226 of the clip 204, such as for example, a plurality of ridges, bars, bumps, teeth, or a pad of material with a higher coefficient of friction than the surrounding material of the housing 302. In operation, the worker or other user may manipulate the cartridge 300 into and out of the receivers 104, 204 described herein by interacting with the grip portion 320 and removably inserting the cartridge 300 in the respective slots 120, 214 of the receivers 104, 204 until the latching device 318 of the cartridge 300 interfaces with the respective latching device 148, 228 of the receivers 104, 204 to secure the cartridge 300 in, or remove the cartridge 300 from, the receivers 104, 204, as appropriate.

FIG. 7 is an embodiment of an electronic system or controller 322 of the detector unit 300 represented in block diagram or schematic form. As further described below, the controller 322 is suitable for executing or otherwise performing at least some embodiments or techniques described herein with respect to the detector unit or cartridge 300. The physical or hardware aspects of the controller 322 may be located internal to the housing 302 of the cartridge 300 (FIG. 6A) with the controller 322 communicatively coupled to at least the input 306 and the circuit board 308 of the cartridge 300 (FIG. 6A), among other devices.

The controller 322 includes a processor 324, for example a microprocessor, digital signal processor, programmable gate array (PGA) or application specific integrated circuit (ASIC). The controller 322 includes one or more non-transitory storage mediums, for example read only memory (ROM) 326, random access memory (RAM) 328, Flash memory (not shown), or other physical computer- or processor-readable storage media in communication with the processor 324. The non-transitory storage mediums may store instructions and/or data used by the processor 324 and the controller 322 generally, for example an operating system (OS) and/or applications. The instructions as executed by the processor 324 may execute logic to perform the functionality of the various implementations or techniques of the devices and systems described herein, including, but not limited to, receiving signals from the sensors described herein, and determining, based on the signals, a type of detected event, whether to issue a notification or warning, whether to alert or other instructions an external device or broader WSS system, and executing such warnings or notifications, among others.

The controller 322 may include one or more sensors 330, such as a current sensor 330A, a voltage sensor 330B, an ambient light sensor 330C, and an accelerometer 330D, among others. For example, the controller 322 may further include gas sensors, GPS receivers and/or transmitters and/or transceivers for providing location information, smoke sensors, oxygen sensors, and biometric sensors. As described in more detail below, the sensors 330 send signals, instructions, or other data to the processor 324 based on a detected occurrence of certain events in a vicinity of a worker or other user in an environment.

The controller 322 may include one or more signaling devices or status indicators 332, such as a lighting element or light array 332A and a speaker 332B. The light array 332A may include at least one lighting element or may include a plurality of light emitting diodes or LEDs arranged in an array ranging from single rows and columns to multiple rows and columns. In an embodiment, the light array 332A includes at least four LEDs disposed on a bottom side of the hardware of the controller 322 and configured to emit light generally through the transparent strip 316A (FIG. 6B) and into a line of sight of a worker or other user. In at least some embodiments, each individual lighting element of the light array 332A may be position- and hue-addressable, such as to control the color and state of each element independently of or in conjunction with the other elements of the light array 332A. The speaker 332B may be a buzzer configured to emit sound as well as haptic signals or vibrations. In some embodiments, the controller 322 may include a separate speaker for emitting sound and a haptic device for emitting vibration, such as to change the strength, volume, or other characteristics of either of these signals relative to a buzzer.

The control unit 322 may include a user interface 334 to allow a worker or other user to operate or otherwise provide input to the cartridge 300, or systems 100, 200 described herein, regarding the operational state or condition of the cartridge 300 and/or the systems 100, 200. In an embodiment, the user interface 334 is implemented at least as the input control device 314 of the cartridge 300 described in FIG. 6A. Additionally, the user interface 334 may include a number of user actuatable controls accessible from the cartridge 300. For example, the user interface 334 may include a number of switches or keys operable to turn the cartridge 300 ON and OFF and/or to set various operating parameters of the cartridge 300, such as sensor sensitivity, operation and control of a start-up or test mode, and operation and control of security functionality independently of or in conjunction with an external device, such as a personal computing device.

In some embodiments, the user interface 334 may include a display, for instance a touch panel display. The touch panel display (e.g., LCD or LED with touch sensitive overlay) may provide both an input and an output interface for the worker or other user. The touch panel display may present a graphical user interface, with various user selectable icons, menus, check boxes, dialog boxes, and other components and elements selectable by the end user to set operational states or conditions of the cartridge 300. The user interface 334 may also include one or more auditory transducers, for example one or more speakers and/or microphones. Such may allow audible alert notifications or signals to be provided to a worker or other user as a result of manual interaction with the user interface 334. Such may additionally, or alternatively, allow a worker or other user to provide audible commands or instructions. The user interface 334 may include additional components and/or different components than those illustrated or described, and/or may omit some components.

The switches and keys or the graphical user interface may, for example, include toggle switches, a keypad or keyboard, rocker switches or other physical actuators of the type described herein. The switches and keys or the graphical user interface 334 may, for example, allow an end user to turn ON the cartridge 300, start or end a test or start-up mode, communicably couple or decouple to remote accessories and programs, access, transmit, or process data, activate or deactivate sensors 330, signaling devices or status indicators 332, and select particular signaling schemes associated with the status indicators 332.

The controller 322 includes a communications sub-system 336 that may include one or more communications modules or components which facilitate communications with various components of one or more external devices, such as a personal computing device, mobile device, server, lighting device 102 (FIG. 1A), and/or WSS system, among others. The communications sub-system 336 may provide wireless or wired communications to the one or more external devices and may include wireless receivers, wireless transmitters and/or wireless transceivers to provide wireless signal paths to the various remote components or systems of the one or more paired devices. The communications sub-system 336 may, for example, include components enabling short range (e.g., via Bluetooth®, BLE (“Bluetooth® low energy”), near field communication (NFC), or radio frequency identification (RFID) components and protocols) or longer range wireless communications (e.g., over a wireless LAN, Low-Power-Wide-Area Network (LPWAN), satellite, or cellular network) and may include one or more modems or one or more Ethernet or other types of communications cards or components for doing so. The communications sub-system 336 may include one or more bridges or routers suitable to handle network traffic including switched packet type communications protocols (TCP/IP), Ethernet or other networking protocols.

The controller 322 further includes a power interface manager 338 that manages supply of power from a power source 340 to the various components of the controller 322 and the cartridge 300. The power interface manager 338 is coupled to the processor 324 and the power source 338. Alternatively, in some implementations, the power interface manager 338 can be integrated in the processor 324. The power source may include an external power supply, or a rechargeable or replaceable battery power supply, among others. The power interface manager 338 may include power converters, rectifiers, buses, gates, circuitry, etc. in some embodiments. In particular, the power interface manager 338 can control, limit, and/or restrict the supply of power from the power source 340 based on the various operational states of the cartridge 300, as described in more detail below.

In an embodiment, the controller 322 stores instructions, such as in ROM 326, RAM 328, and/or flash memory corresponding to a “low power mode” or “battery saving mode” and “a normal power mode” or “normal risk mode,” as well as a “high power mode” or “high risk mode,” among other possibilities. The instructions are executed by the processor 324, which sends one or more signals, data, and/or information to the power interface manager 338 to vary the power from the power source 340 to aspects of the controller 322 according to the mode. For example, the “lower power mode” may include instructions to increase the time between detection cycles by the sensors 330, such as when the worker or other user is in an area of decreased risk of occupational hazards at a worksite. The “normal power mode” may include instructions for the sensors 330 to operate according to a standard or regular selected detection cycle. Similarly, the “high power mode” may include instructions for decreasing the time between detection cycles. The selection of the mode may in some cases be initiated by the lighting device 102, such as based on geographical location data of the worker or other user provided by the GPS chipset of the lighting device 102 indicating the worker or other user is in a high risk area of the worksite. In addition, the selection of the mode may be initiated by a manual input or other interaction with the user interface 334 of the controller 322 of a respective detector unit 300 or another computing device in communication with the detector unit 300.

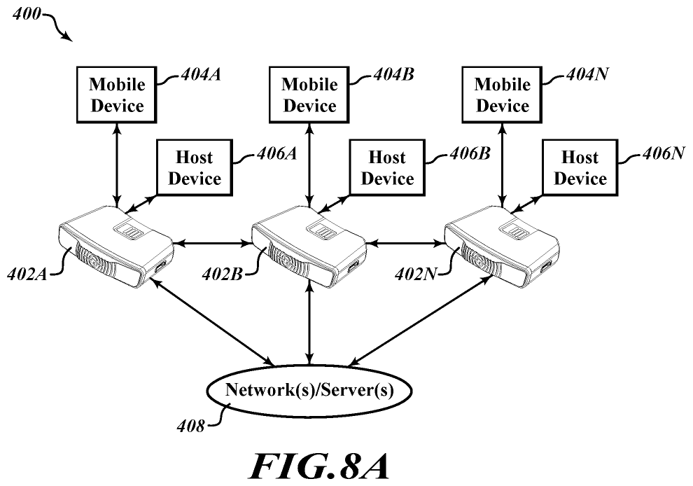

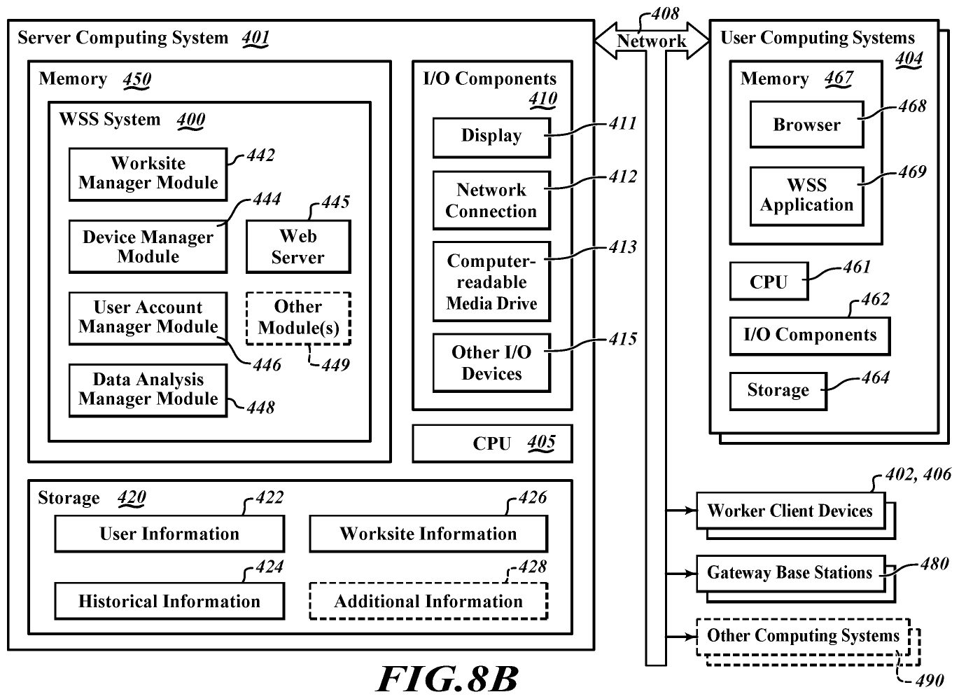

In some embodiments or implementations, the instructions and/or data stored on the non-transitory storage mediums that may be used by the processor 324 and the controller 322 generally, such as, for example, ROM 326, RAM 328 and Flash memory (not shown), includes or provides an application program interface (“API”) that provides programmatic access to one or more functions of the controller 322. For example, such an API may provide a programmatic interface to control one or more operational characteristics of the cartridge 300, including, but not limited to, one or more functions of the user interface 334, processing and/or storing and/or transmitting the data received from the sensors 330, controlling a sensitivity of the sensors 330, and/or defining one or more signaling schemes for the status indicators 332, among others. Such control may be invoked by one of the other programs, other remote device or system, such as of the type described with reference to FIG. 8A and FIG. 8B, or some other module. In this manner, the API may facilitate the development of third-party software, such as various different user interfaces and control systems for other devices, plug-ins, and adapters, and the like to facilitate interactivity and customization of the operation and devices within the cartridge 300.

In an embodiment, components or modules of the controller 322 and other devices within the cartridge 300 and systems 100, 200 described herein are implemented using standard programming techniques. For example, the logic to perform the functionality of the various embodiments or techniques described herein may be implemented as a “native” executable running on the controller 322, e.g., microprocessor 324, along with one or more static or dynamic libraries. In other embodiments, various functions of the controller 322 may be implemented as instructions processed by a virtual machine that executes as one or more programs whose instructions are stored on ROM 326 and/or RAM 328. In general, a range of programming languages known in the art may be employed for implementing such example embodiments, including representative implementations of various programming language paradigms, including but not limited to, object-oriented (e.g., Java, C++, C#, Visual Basic.NET, Smalltalk, and the like), functional (e.g., ML, Lisp, Scheme, and the like), procedural (e.g., C, Pascal, Ada, Modula, and the like), scripting (e.g., Perl, Ruby, Python, JavaScript, VBScript, and the like), or declarative (e.g., SQL, Prolog, and the like).

In a software or firmware implementation, instructions stored in a memory configure, when executed, one or more processors of the controller 322, such as microprocessor 324, to perform the functions of the controller 322. The instructions cause the microprocessor 324 or some other processor, such as an I/O controller/processor, to process and act on information received from one or more sensors or other external devices to provide the detection system functionality and techniques described herein.

The embodiments or implementations described above may also use well-known or other synchronous or asynchronous client-server computing techniques. However, the various components may be implemented using more monolithic programming techniques as well, for example, as an executable running on a single microprocessor, or alternatively decomposed using a variety of structuring techniques known in the art, including but not limited to, multiprogramming, multithreading, client-server, or peer-to-peer (e.g., Bluetooth®, NFC or RFID wireless technology, mesh networks, etc., providing a communication channel between the devices within the cartridge 300 and/or between cartridges 300 and/or between cartridges 300 and a WSS system), running on one or more computer systems each having one or more central processing units (CPUs) or other processors. Some embodiments may execute concurrently and asynchronously, and communicate using message passing techniques. Also, other functions could be implemented and/or performed by each component/module, and in different orders, and by different components/modules, yet still achieve the functions of the controller 322.

In addition, programming interfaces to the data stored on and functionality provided by the controller 322, can be available by standard mechanisms such as through C, C++, C#, and Java APIs; libraries for accessing files, databases, or other data repositories; scripting languages; or Web servers, FTP servers, or other types of servers providing access to stored data. The data stored and utilized by the controller 322 and overall detection devices and systems may be implemented as one or more database systems, file systems, or any other technique for storing such information, or any combination of the above, including implementations using distributed computing techniques.

Different configurations and locations of programs and data are contemplated for use with techniques described herein. A variety of distributed computing techniques are appropriate for implementing the components of the illustrated embodiments in a distributed manner including but not limited to TCP/IP sockets, RPC, RMI, HTTP, and Web Services (XML-RPC, JAX-RPC, SOAP, and the like). Other variations are possible. Other functionality could also be provided by each component/module, or existing functionality could be distributed amongst the components/modules within the cartridge 300 in different ways, yet still achieve the functions of the controller 322 and the cartridge 300.

Furthermore, in some embodiments or implementations, some or all of the components of the controller 322 and components or other devices within the cartridge 300 may be implemented or provided in other manners, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), and the like. Some or all of the system components and/or data structures may also be stored as contents (e.g., as executable or other machine-readable software instructions or structured data) on a computer-readable medium (e.g., as a hard disk; a memory; a computer network, cellular wireless network or other data transmission medium; or a portable media article to be read by an appropriate drive or via an appropriate connection, such as a DVD or flash memory device) so as to enable or configure the computer-readable medium and/or one or more associated computing systems or devices to execute or otherwise use, or provide the contents to perform, at least some of the described techniques.

In an embodiment, the controller 322 is configured to detect whether a current is active in an environment in a vicinity of a worker or other user. Except as otherwise provided herein, the “vicinity” of a worker or other user may include distances of 10 feet or less, 20 feet, 30 feet, 40 feet, 50 feet, 60 feet, 70 feet, 80 feet, 90 feet, or 100 feet or more from the worker or other user, inclusive of intervening and limit values, and can be a selected threshold from the above ranges. In particular, the current sensor 330A of the controller 322 may be provided in the form of a magnetometer that measures magnetic field and/or magnetic dipole moment, or other data, signals and/or information in the environment, and identifies signals within the collected information in a range of 50 to 60 Hz (“hertz”) or more or less. The current sensor 330A may have a selected detection cycle frequency or schedule that may be modified in different modes, as above, and that can be the same or different than the other sensors 330. Except as other indicated herein, the detection cycle frequency of the sensors 330 may be less than 0.1 seconds, 0.2 seconds, 0.3 seconds, 0.4 seconds, 0.5 seconds, 0.6 seconds, 0.7 seconds, 0.8 seconds, 0.9 seconds, 1 second, or 2 seconds or more including intervening and limit values. By sampling information in the environment at a selected schedule and targeting a selected frequency range of signals, the current sensor 330A can determine that an active alternating current (AC) is flowing in the environment and can therefore reduce false readings associated with magnets and/or magnetic fields not otherwise associated with an active current.

The controller 322 is further configured to detect voltage fields or an active alternating current voltage signal in a vicinity of worker or other user in an environment with the voltage sensor 330B. The detection of the voltage or voltage fields may be implemented by the voltage sensor 330B in the form factor of an antenna or other device that measures capacitance to ground in the vicinity of the worker or other user and looks for a selected frequency in that signal, such as a frequency in the range above, to determine whether there is an active voltage in an environment. In an embodiment, the voltage sensor 330B may be configured to measure capacitance with at least two different amplifications to enable the voltage sensor 330B to detect a wide range of voltages, including, for example, 240V to 10,000 C or 100,000V, over a wide range of distances, including, for example, inches away to many feet away. This is advantageous in enabling the device to detect both low voltages and high voltages in the same cartridge 300 with good accuracy over a significant range of distances. In particular, a first amplification (low amplification) may correspond to or be used for detecting low voltages on the order of 240V when a worker is particularly close to the source or for detecting high voltages on the order of 10,000V or 100,000V when about ten feet away, and a second amplification (high amplification) may correspond to or be used for detecting low voltages on the order of 240V when the worker is several feet away from the source or for detecting high voltages on the order of 10,000V or 100,000V when about 50 feet or more away from the source. One or more frequency filtering techniques may be employed to assist in targeting the desired frequency signal(s).

In an embodiment, the cartridge 300 may have different operational modes or other settings configurable by the worker or other user, either through direct manual input on the cartridge 300 or through an external device in communication with the cartridge 300, corresponding to the measurement amplifications above. Thus, for example, a worker or other user, such as a site supervisor, may change the settings and operational characteristics of the cartridge 300 to be more or less sensitive by adjusting the threshold or thresholds at which an alert is generated. In such a situation, the worker or other user may not receive an alert for known active voltages below a selected threshold but will receive a signal or warning in response to detection of a higher voltage to reduce the likelihood that the cartridge 300 will constantly provide alerts for known low voltages that are of less concern to the worker or other user, while maintaining detection functionality for dangerously high and potentially life threatening voltages. In some embodiments, a worker or other user may be presented with an interface to select between a “low voltage” mode and a “high voltage” mode and in response a different sensitivity, or threshold level, may be established based on the selection. Thus, the threshold may be adjusted directly or indirectly by the worker or other user.

In an embodiment, the RAM 326, ROM 328, and/or flash memory of the controller 322 of the cartridge 300 may store further instructions that can be executed by the processor 324 as logic corresponding to a threshold adaptive mode. The threshold adaptive mode may change one or more thresholds of the sensors 330 of the controller 322 based on a change in a percentage of strength and/or magnitude of a detected field over time.

For example, if a worker or other user associated with the cartridge 300 moves into a cautionary zone where the current and/or voltage detected by the sensors 330A, 330B is at a first level at or exceeding a “caution” threshold of the sensors 330A, 330B but below a “high alert” threshold of the sensors 330A, 330B, the processor 324 may execute instructions to provide one or more cautionary signals (e.g., a slow flashing yellow light) with the signaling devices 332. With threshold adaptive mode active, the processor 324 executes instructions to terminate at least some aspects of the cautionary signals (e.g., terminate the slow flashing of the yellow light and display static yellow light) after a selected period of time, such as five to ten seconds in a non-limiting example, or in response to a user selection to terminate or partially terminate the cautionary signals. Then the same cautionary signals are disabled or prevented until, for example, the worker or other user associated with the cartridge 300 moves into an area within the cautionary zone where the current and/or voltage detected by the sensors 330A, 330B increases by at least an established percentage (e.g., 5%, 10%, or 20%) over a select period of time relative to the first level, or a new line is activated causing the current and/or voltage detected by the sensors 330A, 330B to increase by at least the established percentage (e.g., 5%, 10%, or 20%) over the select period of time relative to the first level. When the user moves into or is otherwise subjected to such an area within the cautionary zone where the current and/or voltage detected by the sensors 330A, 330B increases by at least the established percentage (e.g., 5%, 10%, or 20%) over the first level, then the same cautionary signals (e.g., slow flashing yellow light) may be reissued again or a variation of the cautionary signals (e.g., faster flashing signal and/or different tone of yellow signal) may be issued.

Similarly, if a worker or other user associated with the cartridge 300 moves into or is otherwise subjected to a high alert zone where the current and/or voltage detected by the sensors 330A, 330B is at a second level at or exceeding the “high alert” threshold of the sensors 330A, 330B, the processor 324 may execute instructions to provide one or more high alert signals (e.g., a fast flashing red light) with the signaling devices 332. With threshold adaptive mode active, the processor 324 executes instructions to terminate at least some aspects of the high alert signals (e.g., terminate the fast flashing of the red light and display static red light) after a selected period of time, such as five to ten seconds in a non-limiting example, or in response to a user selection to terminate or partially terminate the high alert signals. Then the same high alert signals are disabled or prevented until the worker or other user associated with the cartridge 300 moves into or is otherwise subjected to an area within the high alert zone where the current and/or voltage detected by the sensors 330A, 330B increases by at least an established percentage (e.g., 5%, 10%, or 20%) over a select period of time relative to the second level. When the user moves into or is otherwise subjected to such an area within the high alert zone where the current and/or voltage detected by the sensors 330A, 330B increases by at least the established percentage (e.g., 5%, 10%, or 20%) over the second level, then the same high alert signals (e.g., fast flashing red light) may be reissued again or a variation of the high alert signals (e.g., faster flashing signal and/or different tone of red signal) may be issued.

Another aspect of the threshold adaptive mode is that if the signal strength decreases for an amount of time, for example, because the user moves away from the voltage or current source, then the controller may re-establish the 5%, 10%, 15%, etc. increasing thresholds to enable alerts to be issued again as the user is subjected to increases in the signal strength for an amount of time, for example, when the user moves back toward the voltage or current source.

In an embodiment, the cautionary signals may include one or more of an animated light pattern in a yellow color emitted by the light array 332A, a selected sound emitted from the speaker 332B, and a haptic signal from the speaker 332B and/or buzzer. The processor 324 may execute instructions to terminate the audible and/or haptic signals after the selected period of time, and also terminate animation or other characteristics of the lighting scheme associated with the light array 332A. The processor 324 may also execute instructions to terminate emission of light with at least some individual elements of the light array 332A, such as half of the individual light elements of the array 332A while the remaining light elements of the array 332A remain active. At the same time, the processor 324 may execute instructions to raise an alert threshold for a detected current and/or voltage from the “caution” threshold associated with the caution zone to an established percentage increase (e.g., 5%, 10%, or 20%) over the first level (i.e., the processor 324 increases the alert threshold of the sensors 330 to a value that is an established percentage increase (e.g., 5%, 10%, or 20%) over the detected value when the cautionary signals are terminated) so that the cartridge 300 does not continuously issue cautionary signals after the selected period of time, assuming that changes exceeding the buffer of the established percentage have not occurred.

Similarly, in an embodiment, the high alert signals may include one or more of an animated light pattern in a red color emitted by the light array 332A, a selected sound emitted from the speaker 332B, and a haptic signal from the speaker 332B and/or buzzer. The processor 324 may execute instructions to terminate the audible and/or haptic signals after the selected period of time, and also terminate animation or other characteristics of the lighting scheme associated with the light array 332A. The processor 324 may also execute instructions to terminate emission of light with at least some individual elements of the light array 332A, such as half of the individual light elements of the array 332A while the remaining light elements of the array 332A remain active. At the same time, the processor 324 may execute instructions to raise an alert threshold for a detected current and/or voltage from the “high alert” threshold associated with the high alert zone to an established percentage increase (e.g., 5%, 10%, or 20%) over the second level (i.e., the processor 324 increases the alert threshold of the sensors 330 to a value that is an established percentage increase (e.g., 5%, 10%, or 20%) over the detected value when the high alert signals are terminated) so that the cartridge 300 does not continuously issue high alert signals after the selected period of time, assuming that changes exceeding the buffer of the established percentage have not occurred.

Thus, the worker or other user will receive the cautionary signals when the detected current and/or voltage exceeds the “caution” threshold, and at least some part of the cautionary signals will terminate after a short period of time. An alert threshold is then adjusted to an established percentage increase over the detected level and some aspects of the cautionary signals may remain active, such as at least a limited lighting scheme (e.g., static yellow light of reduced intensity), to indicate that the worker or other user remains within the caution zone but that the voltage and/or current have not changed beyond the buffer of the established percentage.

Similarly, the worker or other user will receive the high alert signals when the detected current and/or voltage exceeds the “high alert” threshold, and at least some part of the high alert signals will terminate after a short period of time. An alert threshold is then adjusted to an established percentage increase over the detected level and some aspects of the high alert signals may remain active, such as at least a limited lighting scheme (e.g., static red light of reduced intensity), to indicate that the worker or other user remains within the high alert zone but that the voltage and/or current have not changed beyond the buffer of the established percentage.

Changes in detected current and/or voltage may occur as a result of the worker or other user associated with the cartridge 300 moving closer to the source of the current and/or voltage, as well as if power is restored to previously inactive lines. If the determined percentage change in magnitude of the detected current and/or voltage exceeds the established percentage associated with the new alert threshold, the processor 324 executes instructions to provide one or more additional signals, which may be the same or similar to the cautionary and high alert signals except for the signaling scheme (i.e., different colors, sounds, haptic outputs). The processor 324 then executes instructions to likewise terminate at least some aspects of these additional signals, and adjusts the alert threshold of the sensors 330A, 330B to an established percentage increase over the detected level, as described above. This process can be repeated in an iterative process to reduce the active alerts or warnings provided to the worker or other user over time within each of the caution zone and high alert zone when they are aware of active voltages and/or currents in an environment.

Notably, while aspects of the threshold adaptive mode are described above in the context of two distinct alert zones associated with respective alert thresholds, namely, the caution zone and the high alert zone, it is appreciated that one or more embodiments may include no zones, one zone or more than the two zones. For example, one embodiment may include a low-level alert zone, a medium-level alert zone, and a high-level alert zone with respective alert thresholds for each.

Additionally, in some instances, multiple alert types may be presented simultaneously. For example, a cautionary current alert may be shown on some of a plurality of light elements of the light array 332A, while a cautionary voltage alert may be shown simultaneously on others of the plurality of light elements of the light array 332A. In other instances, multiple alert types may be presented alternately on the light array 332A.

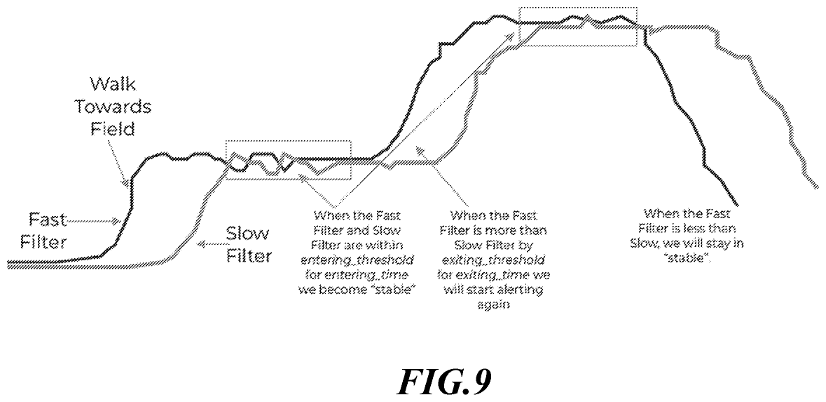

In an embodiment, the threshold adaptive mode is activated or turned ON and/or OFF via a manual input on the user interface 334 of the controller 322, including but not limited to a specific button manipulation scheme on the input control device 314 of the cartridge 300 (FIG. 6A), such as pressing the input control 314 in a selected sequence, frequency, or duration. In yet further embodiments, the threshold adaptive mode may be activated or terminated (i.e., turned ON and/or OFF) via a manual input on the lighting device 102 and/or a manual input or selection on the other devices 342 in communication with the controller 322, such as for example, input or selection on a personal computing device or a mobile device in communication with the controller 322 of the cartridge 300 (FIG. 6A). The threshold adaptive mode will be described in more detail with reference to FIG. 9.

The ambient light sensor 330C may be implemented as a photodetector configured to sense the amount of ambient light present in the environment in the vicinity of the worker or other user. Such information may then be provided to the processor 324 and the controller 322 generally for modifying the signaling or notification scheme, among other functionality.

The accelerometer 330D may be implemented in any of the hardware components discussed herein, including but not limited to a chip or chipset or integrated into a chip or chipset, and is configured to detect a “fall event.” Unless the context dictates otherwise, a “fall event” is defined as a selected threshold rate of acceleration over a period of time, such as an acceleration that approaches or exceeds the acceleration of gravity (i.e., 9.8 m/s² or between 6 m/s² to 10 m/s² or more) over a period of time of 0.25 seconds or less, 0.5 seconds, 1 second, 2 seconds, or more. In such a way, the accelerometer is configured to detect both fall events that may occur due to a fall from an elevated position (i.e., a free fall at or near the acceleration of gravity over a longer period) as well as a fall event associated with a worker or other user being knocked over by an external force, which may in some cases exceed the rate of acceleration of gravity over a shorter period. In addition, the accelerometer 330D may be used for other functions, such as, for example, to detect motion of the worker or other user and/or to detect when the host device is idle. This could help distinguish between events such as a worker or other user moving closer to or away from a current or voltage source and an idle worker or other user experiencing a voltage or current signal increase from a new source turning on or a power line falling. Accordingly, use of the accelerometer 330D is not limited to only detecting a fall event.

The input 306 is in communication with the controller 322, and particularly at least the power interface manager 338 and the power source 340 for providing power from an external source to recharge the power source 340 under the supervision of the power interface manager 338. For example, the power interface manager 338 may determine the rate of charging based on the input voltage and current as well as when to start and stop charging of the power source 340, among other functionality. In an embodiment, the input 306 is also in communication with the processor 324 and the communications subsystem 336 such as, for example, to provide signals, instructions, and/or other data to one or more external devices 342. Similarly, the lighting device 102 may be in communication with the controller 322 for providing the additional signaling and interfacing functionality of the embodiments of the detection systems 100, 200 described herein. Other sensors of the type described herein may interface with the controller 322 similarly, regardless of whether such sensors are provided in the cartridge 300 or in an external device 342 that interfaces with the cartridge 300 and the controller 322.

When one or more of the current and/or voltage sensors 330A, 330B and accelerometer 330D detect occurrence of an event, as above, the sensors 330A, 330B, 330D send a signal, instructions and/or other data to the processor 324. Upon receipt of the information from the sensors 330A, 330B, 330D, the processor 324 may execute instructions stored in the ROM 326, RAM 328, and/or flash memory to change at least one status indicator of the cartridge 300, such as a characteristic of the light array 332A and/or the speaker 332B. In particular, the ROM 326, RAM 328, and/or flash memory may store instructions for execution by the processor 324 corresponding to predefined or selected notification or warning schemes, including but not limited to: one or more color combinations for the light array 332A as well as for each individual light element of the array 332A; one or more pulsing or animated lighting patterns for the light array 332A; sounds of different length, strength or volume, and frequency from the speaker 332B; and different haptic alert patterns and strengths in embodiments where the speaker 332B is implemented as a buzzer or the cartridge otherwise includes a vibration device, among others.

The ambient light sensor 330C may also impact the notification scheme executed by the controller 322 depending on conditions in the environment. For example, if the ambient light sensor 330C detects that ambient light is high or bright, the light sensor 330C may transmit that information to the processor 324, which in turn, executes instructions for providing audible or haptic alerts with the speaker 332B instead of visual alerts with the light array 332A given that the light array 332A may be difficult to see clearly or may not otherwise provide an effective notification in bright conditions. The opposite may also be true, namely, if the light sensor 330C determines that an environment is dim or has low light, then the processor 324 executes instructions to provide a visual alert with the light array 332A alone, or in conjunction with, alerts from the other status indicators.

The processor 324 may also interface with the communications subsystem 336 and execute instructions stored in the ROM 326, RAM 328, and/or flash memory for transmitting signals, instructions and/or other data from the controller 322 to other external devices 342 that interface with the cartridge 300 and the controller 322. In an embodiment, the communications subsystem 336 transmits signals, instructions, and/or other data to the lighting device 102 for amplification of the signal, such as for, example, to display the lighting scheme on the lighting device 102. The communications subsystem 336 may also receive signals, instructions, and/or data from the external devices 342, such as, for example, an indication of a detected fall event by the external devices 342 or a notification of an emergency situation from a WSS system, where the processor 342 is configured to execute further instructions to change at least one characteristic of the status indicators 332A, 332B based on the received data. The external devices 342 may also be personal computing devices associated with the worker or other user, as described in more detail in FIG. 8A and FIG. 8B. The ROM 326, RAM 328 and/or flash memory may also store or log information regarding events detected by the cartridge 300 over a selected period of time for transmission to an external device 342 for evaluation via wired connection through input 306 and/or wirelessly through the communications subsystem 336, among other data.

FIG. 8A is a schematic diagram providing an overview of an embodiment of network and communication operations for the detection systems and devices of the present disclosure. FIG. 8B is a block diagram of a computing system suitable for executing an embodiment of a system that performs at least some techniques described in the present disclosure, as well as various devices and/or computing systems connected thereto, such as at least some of the techniques described with reference to FIG. 8A. In FIG. 8A and FIG. 8B, the network and communications operations and the computing system may be part of a configurable WSS system 400 for increasing safety and reducing risks at a single worksite or multiple worksites.

Beginning with FIG. 8A, the WSS system 400 may generally include a number of detector units 402, including detector units 402A, 402B, 402N of the type described herein. Unless the context dictates otherwise, the “N” designation in the reference numbers of FIG. 8A and FIG. 8B indicates that a particular set or group may include a selected number or “N” number of systems or devices that may be more or less than the number shown in FIG. 8A. The detector units 402 are in communication with each other, or otherwise interface with each other, such as through the communications subsystem 336 of the controller 322 (FIG. 7) of each detector unit 402. In an embodiment, selected ones of the detector units 402 are grouped together in a subset of the detector units 402 for communication across unique communication channels according to the communication protocols described herein. Such grouping may be selected via manual input on the user interface 334 (FIG. 7) of each detector unit 402, or the detector units 402 may be grouped by an input to, or a selection on, a computing system of the WSS 400 in communication with each of the detector units 402 and described in more detail with reference to FIG. 8B. The detector units 402 in a subset may communicate warnings, notifications, or alerts to each other across the respective communication channel to alert others in the subset of a detected event. In an embodiment, such subsets may be determined by workers or other users operating in the vicinity of each other at a worksite, although the subsets may also be selected according to other factors.

Each of a plurality of mobile devices 404, including mobile devices 404A, 404B, 404N, are in communication with a respective one of the detector units 402A, 402B, 402N. The mobile devices 404 may also be other smart devices and/or personal computing devices. In an embodiment, the mobile devices 404 are in communication with only the respective one of the detector units 402 through any of the communication protocols described herein following a pairing or other setup process to initiate communication and a secure exchange of information, such as via Bluetooth®. Such pairing process may include manual input by a worker or other user on the user interface 334 (FIG. 7) of the detector unit 404 and/or the mobile device 404 to establish a connection between one of the detector units 402 and a respective one of the mobile devices 404. The mobile device 404 may display a graphical user interface (“GUI”) for modifying at least one characteristic of the respective detector unit 402 to which it is paired, such as, for example, a sensitivity of the controller 322 (FIG. 7) of the respective detector unit 402, the subset or communication group of the respective detector unit 402, and/or the signaling scheme characteristics of the respective detector unit 402.

A plurality of host devices 406, including host devices 406A, 406B, 406N, interface with, or are otherwise in communication with, the detector units 402. In an embodiment, the host devices 406 are the lighting device 102 (FIG. 1A), ring signaling device, or other safety equipment or gear of the worker or other user. In an embodiment, each of the host devices 406A, 406B, 406N is in communication with, or interfaces with a single respective one or only a respective one of the detector units 402A, 402B, 402N. The connection between the host devices 406 and the detector units 402 may be a wired or a wireless connection, as described herein. The detector units 402 may send signals, instructions, and/or other data to the host devices 406, such as information pertaining to a detected event, to change at least one characteristic of the corresponding host device 406. The detector units 402 may also receive such information from the host devices 406 in return.

Each mobile device 404 and host device 406 is preferably associated with one worker or other user and only a single respective detector unit 402 worn or carried by the one worker or other user for security and safety purposes. As described herein, certain aspects and settings, among other features, of each detector unit 402 are customizable with manual selections or inputs according to various factors, including but not limited to, the type of work performed by a worker or other user (i.e., low or high risk for occurrence of certain events), experience level of the worker or other user, location on a worksite, and others. Thus, while having a mobile device 404 and/or a host device 406 in communication with more than one detector unit 402 is contemplated herein and achievable by the concepts of the disclosure, such an arrangement may increase risks if settings or other aspects of the respective detector unit 402 are changed by a different user without warning to the worker or other user carrying the respective detector unit 402 and is therefore less preferred. In an embodiment, settings and other aspects of each detector 402 can only be changed by manual input or selection on the mobile device 404 associated with the respective detector unit 402 to which it is paired along with a manual input or interaction with the user interface 334 of the controller 322 (FIG. 7) of the respective detector unit 402.

Each of the detector units 402 is in communication with a network or server system 408, as described in more detail in FIG. 8B. Such system 408 may receive signals, instructions, and/or other information from one or more detector units 402, such as information relating to a detected event, and issue one or more alerts or warnings to selected subset or all of the detector units 402 and/or mobile devices 404 and/or host devices 406, including but not limited to an SMS or text message alert or instructions to change at least one characteristic of the detector units 402 and/or host devices 406, such as a signaling scheme, or otherwise notify supervising or other selected staff regarding the occurrence of a detected event.

FIG. 8B is a block diagram illustrating component-level functionality provided by a plurality of electronic circuits that, when in combined operation, are suitable for performing and configured to perform at least some of the techniques described herein. In the particular implementation depicted, the plurality of electronic circuits is at least partially housed within a server computing system 401 executing an implementation of a WSS system 400. The server computing system 401 includes one or more central processing units (“CPU”) or other processors 405, various input/output (“I/O”) components 410, storage 420, and memory 450, with the illustrated I/O components including a display 411, a network connection 412, a computer-readable media drive 413, and other I/O devices 415 (e.g., keyboards, mice or other pointing devices, microphones, speakers, GPS receivers, etc.). The server computing system 402 and WSS system 400 may communicate with other computing systems via one or more networks 408 (e.g., the Internet, one or more cellular telephone networks, etc.), such as mobile devices 404 (which may also be referred to as user computing systems 404), detector units 402 and/or host devices 406 (which may generally be referred to as worker client devices 402, 406), gateway base stations 480, and other computing systems 490. Some or all of the user computing systems 404 and other computing systems 490 may similarly include some or all of the types of components illustrated for server computing system 401 (e.g., to have a WSS system client application 469 executing in memory 467 of a user computing system 404 in a manner analogous to WSS system 400 in memory 450).

In the illustrated embodiment, an embodiment of the WSS system 400 executes in memory 450 in order to perform at least some of the described techniques, such as by using the processor(s) 405 to execute software instructions of the system 400 in a manner that configures the processor(s) 405 and computing system 401 to perform automated operations that implement those described techniques. As part of such automated operations, the WSS system 450 and/or other optional programs or modules 449 executing in memory 450 may store and/or retrieve various types of data, including in the example database data structures of storage 420. In this example, the data used may include various types of user information in database (“DB”) 422, various types of historical information (such as events detected by a detector unit) in DB 424, various types of worksite information in DB 426, and/or various types of additional information 428, such as various analytical information related to one or more devices or worksites associated with the WSS system.

It will be appreciated that computing system 401 other systems and devices included within FIG. 8B are merely illustrative and are not intended to limit the scope of the present invention. The systems and/or devices may instead each include multiple interacting computing systems or devices, and may be connected to other devices that are not specifically illustrated, including via Bluetooth communication or other direct communication, through one or more networks such as the Internet, via the Web, or via one or more private networks (e.g., mobile communication networks, etc.). More generally, a device or other computing system may comprise any combination of hardware that may interact and perform the described types of functionality, optionally when programmed or otherwise configured with particular software instructions and/or data structures, including without limitation desktop or other computers (e.g., tablets, slates, etc.), database servers, network storage devices and other network devices, smart phones and other cell phones, consumer electronics, wearable devices, biometric monitoring devices, digital music player devices, handheld gaming devices, PDAs, wireless phones, Internet appliances, and various other consumer products that include appropriate communication capabilities. In addition, the functionality provided by the illustrated WSS system 400 may in some embodiments be distributed in various modules. Similarly, in some embodiments, some of the functionality of the WSS system 400 may not be provided and/or other additional functionality may be available. In addition, in certain implementations various functionality of the WSS system may be provided by third-party partners of an operator of the WSS system. For example, data collected by the WSS system may be provided to a third party for analysis and/or metric generation.

It will also be appreciated that, while various items are illustrated as being stored in memory or on storage while being used, these items or portions of them may be transferred between memory and other storage devices for purposes of memory management and data integrity. Alternatively, in other embodiments some or all of the software modules and/or systems may execute in memory on another device and communicate with the illustrated computing systems via inter-computer communication. Thus, in some embodiments, some or all of the described techniques may be performed by hardware means that include one or more processors and/or memory and/or storage when configured by one or more software programs (e.g., the WSS system 400 and/or WSS client software executing on user computing systems 404 and/or worker client devices 402, 406) and/or data structures, such as by execution of software instructions of the one or more software programs and/or by storage of such software instructions and/or data structures. Furthermore, in some embodiments, some or all of the systems and/or modules may be implemented or provided in other manners, such as by consisting of one or more means that are implemented at least partially in firmware and/or hardware (e.g., rather than as a means implemented in whole or in part by software instructions that configure a particular CPU or other processor), including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic devices (CPLDs), etc. Some or all of the modules, systems and data structures may also be stored (e.g., as software instructions or structured data) on a non-transitory computer-readable storage mediums, such as a hard disk or flash drive or other non-volatile storage device, volatile or non-volatile memory (e.g., RAM or flash RAM), a network storage device, or a portable media article (e.g., a DVD disk, a CD disk, an optical disk, a flash memory device, etc.) to be read by an appropriate drive or via an appropriate connection. The systems, modules and data structures may also in some embodiments be transmitted via generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of the present disclosure may be practiced with other computer system configurations.

FIG. 9 is a graphical representation illustrating at least some techniques and concepts of a threshold adaptive mode of embodiments of the detection systems of the present disclosure. As explained above, the threshold adaptive mode may be implemented and/or executed via the hardware of the controller 322 (FIG. 7) and/or the electric systems of the other devices described herein. With reference to FIG. 9 and with continuing reference to FIG. 6A and FIG. 7, the threshold adaptive mode generally relies on the interpretation of data fed through two filters with the controller 322 activating and/or deactivating signaling schemes based on the results of the interpretation. In particular, the threshold adaptive mode may be implemented at least in part based on a “fast” filter and a “slow” filter. The “fast” and “slow” designations may refer to a rate or frequency at which the filters operate or at which data is fed through the filters and/or the rate of movement of the worker or other user, among other possibilities. Both filters may be implemented according to the equation new_average=new_data*alpha+old_average (1−alpha) where alpha is generally a selected value between 0 and 1. The alpha value for the “fast” filter may be the same or different from the alpha value for the “slow” filter. In an embodiment, a different type of filter than “fast” and “slow” filters is selected for processing the data, including at least a trailing average filter, among others.

The threshold adaptive mode also includes two states associated with the cartridge 300. The states may correspond to the data input to and/or output from the filters as well as the signaling schemes executed by the controller 322. Specifically, the threshold adaptive mode may include a “reading is stable and/or decreasing” state and a “reading is increasing” state. In some embodiments, the stable and decreasing conditions are separate states, but are considered together because the resulting signaling scheme to the worker or other user is the same in either state, as explained further below. However, the stable and decreasing states may also correspond to additional and different functionality between these states in some embodiments. The controller may activate and/or deactivate different signaling schemes based on the identified state of the cartridge 300 and/or a determined transition between the above states.

To identify a transition from the “stable/decreasing” state to the “increasing” state, the controller 322 looks for a value equal to “fast_filter_value−slow_filter_value” to be greater than a selected threshold or “exiting_threshold.” In an embodiment, the difference between the fast and slow filter values is identified as a transition between states if the difference is greater than “exiting_threshold” for a selected time (or “exiting_time”), which is generally a selected value in seconds. The “exiting_time” may be a continuous period of time in some embodiments, meaning that the difference between the fast and slow threshold values is continuously greater than the “exiting_threshold” over the “exiting_time” to identify a transition. If the difference between the fast and slow filter values is less than “exiting_threshold” at any point during the “exiting_time,” the controller 322 may reset the timer for the “exiting_time” in some embodiments.

To identify a transition from the “increasing” state to the “stable/decreasing” state, the controller 322 (FIG. 7) looks for the difference between the fast and slow filter values to be less than a threshold or “entering_threshold” for a selected period of time or “entering_time.”

When the controller 322 determines that the system and/or the cartridge 300 is in the “stable/decreasing” state, the controller 322 may execute instructions corresponding to a “Stable UX” and activate a single lighting element of the light array 332A with defined animation, such as a single slow blink with no sounds. Other signaling schemes may also be activated and/or deactivated in association with the “Stable UX.” When the controller 322 determines that the system and/or cartridge 300 has transitioned, as above, from the “stable/decreasing state” to the “increasing state,” the controller 322 may execute instructions for a “Normal UX,” which may include beeping and blinking lights in different colors, such as red and yellow, similar to the description above with reference to FIG. 7. The controller 322 may not execute instructions to provide any signals when the determined voltage and/or current is less than the yellow threshold, but the controller 322 may be evaluating if the cartridge 300 is in the “stable/decreasing” or “increasing” states such that the threshold adaptive mode can be active when the cartridge 300 is close to, but has not passed, the yellow threshold.

The above process is summarized by the graphical representation in FIG. 9, which shows the fast filter readings with a black line and the slow filter readings with a grey line. As the worker or other user moves closer to a voltage and/or current field, the outputs from the filters may generally increase in magnitude and a difference between the values likewise increases. When the difference between the filter values exceeds the exiting_threshold over the exiting_time at a first instance associated with the “caution” threshold, the controller 322 may execute instructions to activate the “caution” signaling scheme. Then, if the worker or other user remains at a relatively constant position with respect to the voltage and/or current field, the fast and slow filter values begin to align. When the difference between the fast and slow thresholds are within “entering_threshold” for the “entering_time,” the controller 322 executes instructions associated with the “stable” state above to deactivate the “caution” signals and activate the “stable” signals described above. As the worker or other user moves even closer to the voltage and/or current field, the fast and slow filter values will increase again and, if the values pass the “high” threshold, the controller 322 executes instructions to deactive the “stable” signals and activate the “high” threshold signals. Then, if the worker or other user stops moving closer to the current and/or voltage field at a second instance, the process above is repeated. More specifically, the controller 322 deactivates the “high” threshold signals and again activates “stable” signals, although the color of light or other characteristics of the signals may be different to provide an indication that the worker or other user has passed the relevant threshold and is now closer to the detected voltage and/or current field and is therefore at a higher risk. As the worker or other user moves away from the current and/or voltage field, the fast filter is less than the slow filter, and the controller 322 executes instructions to maintain the “stable” signaling scheme. Thus, the output signaling scheme to the worker or other user may be the same in the stable and decreasing states in some embodiments.

From the foregoing it will be appreciated that, although specific embodiments have been described herein for purposes of illustration, various modifications may be made without deviating from the spirit and scope of the invention. Accordingly, the invention is not limited except as by corresponding claims and the elements recited by those claims. In addition, while certain aspects of the invention may be presented in certain claim forms at certain times, the inventors contemplate the various aspects of the invention in any available claim form.

The above description of illustrated embodiments, including what is described in the Abstract, is not intended to be exhaustive or to limit the implementations to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied outside of the detection system and device context, and are not limited to the example detection systems, methods, and devices generally described above.

For instance, the foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one implementation, the present subject matter may be implemented via Application Specific Integrated Circuits (ASICs). However, those skilled in the art will recognize that the implementations disclosed herein, in whole or in part, can be equivalently implemented in standard integrated circuits, as one or more computer programs executed by one or more computers (e.g., as one or more programs running on one or more computer systems), as one or more programs executed by on one or more controllers (e.g., microcontrollers) as one or more programs executed by one or more processors (e.g., microprocessors), as firmware, or as virtually any combination thereof, and that designing the circuitry and/or writing the code for the software and or firmware would be well within the skill of one of ordinary skill in the art in light of the teachings of this disclosure.

When logic is implemented as software and stored in memory, logic or information can be stored on any computer-readable medium for use by or in connection with any processor-related system or method. In the context of this disclosure, a memory is a computer-readable medium that is an electronic, magnetic, optical, or other physical device or means that contains or stores a computer and/or processor program. Logic and/or the information can be embodied in any computer-readable medium for use by or in connection with an instruction execution system, apparatus, or device, such as a computer-based system, processor-containing system, or other system that can fetch the instructions from the instruction execution system, apparatus, or device and execute the instructions associated with logic and/or information.

In the context of this specification, a “computer-readable medium” can be any element that can store the program associated with logic and/or information for use by or in connection with the instruction execution system, apparatus, and/or device. The computer-readable medium can be, for example, but is not limited to, an electronic, magnetic, optical, electromagnetic, infrared, or semiconductor system, apparatus or device. More specific examples (a non-exhaustive list) of the computer readable medium would include the following: a portable computer diskette (magnetic, compact flash card, secure digital, or the like), a random access memory (RAM), a read-only memory (ROM), an erasable programmable read-only memory (EPROM, EEPROM, or Flash memory), a portable compact disc read-only memory (CDROM), digital tape, and other nontransitory media.

Many of the methods described herein can be performed with variations. For example, many of the methods may include additional acts, omit some acts, and/or perform acts in a different order than as illustrated or described.

Thus, in one or more embodiments, a detection system may be summarized as including: a detector unit wearable by a user, including a status indicator and a controller in electronic communication with the status indicator. The controller includes a memory configured to store instructions and at least one processor configured to execute the instructions to detect whether a voltage is active in an environment in a vicinity of the user, including measuring capacitance at different amplifications, and change at least one characteristic of the status indicator in response to detecting the voltage.

The memory is configured to store further instructions and the at least one processor is configured to execute the further instructions to detect whether an electrical current is active in the environment in a vicinity of the user, and change at least one characteristic of the status indicator in response to detecting the current.

The memory is configured to store further instructions and the at least one processor is configured to execute the further instructions to detect whether a fall event associated with the user of the detector unit has occurred, and change at least one characteristic of the status indicator in response to detecting the occurrence of the fall event.

The detection system may further include a receiver having a detector unit slot with the detector unit provided in the form of a functional detector cartridge that is removably insertable in the detector unit slot of the receiver.

The receiver is configured to be removably coupleable to headgear or other equipment worn by the user of the detector unit and includes a latching device within the detector unit slot to selectively secure the functional detector cartridge within the detector unit slot of the receiver.

The detection system may further include a lighting device wearable by the user of the detector unit and the receiver is provided in the form of an adapter that is removably coupleable to the lighting device to enable the functional detector cartridge to interface with the lighting device.

The adapter includes one or more electrical contacts and is configured to support the functional detector cartridge in an electrically connected manner to an electrical system of the lighting device to receive electrical power from the lighting device and/or to enable the functional detector cartridge to receive one or more signals from the lighting device and/or to transmit one or more signals to the lighting device via the one or more electrical contacts of the adapter.

The adapter further includes an electrical connector for interfacing with an electrical system of the lighting device and at least one electrical contact for interfacing with an electrical system of the functional detector cartridge. The lighting device has a corresponding electrical connector configured to interface with the electrical connector of the adapter when the adapter is secured to the lighting device. The functional detector cartridge includes at least one electrical contact configured to interface with the at least one electrical contact of the adapter to provide power from the lighting device to the functional detector cartridge and/or to enable the functional detector cartridge to receive one or more signals from the lighting device and/or to transmit one or more signals to the lighting device.

The functional detector cartridge may be in communication with the lighting device and the memory may be configured to store further instructions, the at least one processor configured to execute the further instructions to change at least one characteristic of the lighting device in response to detecting the voltage.

The detector unit is in communication with a lighting device that is remote from the detector unit, and the memory is configured to store further instructions, the at least one processor configured to execute the further instructions to change at least one characteristic of the lighting device in response to detecting the voltage.

The detector unit may also be configured to receive power from a lighting device to which the detector unit is coupleable.

One or more embodiments of a detection system may be summarized as including a detector unit wearable by a user, including a status indicator and a controller in electronic communication with the status indicator, the controller including a memory configured to store instructions and at least one processor configured to execute the instructions to: detect whether a voltage is active in an environment in a vicinity of the user; detect whether an electrical current is active in the environment in the vicinity of the user; detect whether a fall event associated with the user of the detector unit has occurred; and change at least one characteristic of the status indicator in response to detecting at least one of the voltage, the electrical current, and the fall event.

The detection system may further include a receiver removably coupleable to headgear or other equipment of the user and configured to removably receive the detector unit.

The receiver includes a detector unit slot, the detector unit provided in the form of a functional detector cartridge that is removably insertable in the detector unit slot of the receiver.

The receiver further includes a latching device within the detector unit slot to selectively secure the functional detector cartridge within the detector unit slot of the receiver.

The detector unit is configured to receive power from a lighting device in communication with the detector unit.

The detection system may further include a lighting device wearable by the user and an adapter removably coupleable to the lighting device and configured to removably receive the detector unit to secure the detector unit to the lighting device.

The detector unit is electrically connected to the lighting device and the lighting device is configured to provide power to the form factor via the adapter and/or to enable the detector unit to receive one or more signals from the lighting device and/or to transmit one or more signals to the lighting device via the adapter.

The adapter includes an electrical connector for interfacing with an electrical system of the lighting device and at least one electrical contact for interfacing with an electrical system of the functional detector cartridge. The lighting device has a corresponding electrical connector configured to interface with the electrical connector of the adapter when the adapter is secured to the lighting device. The functional detector cartridge includes at least one electrical contact configured to interface with the at least one electrical contact of the adapter to provide power from the lighting device to the functional detector cartridge and/or to enable the functional detector cartridge to receive one or more signals from the lighting device and/or to transmit one or more signals to the lighting device.

The detector unit is in communication with the lighting device and the memory is configured to store further instructions, the at least one processor configured to execute the further instructions to change at least one characteristic of the lighting device in response to detecting at least one of the voltage, the electrical current, and the fall event.

The detector unit is in communication with a lighting device and the memory is configured to store further instructions, the at least one processor configured to execute the further instructions to change at least one characteristic of the lighting device in response to detecting at least one of the voltage, the electrical current, and the fall event.

The memory is configured to store further instructions and the at least one processor is configured to execute the further instructions to measure capacitance of the voltage at different amplifications.

One or more embodiments of a detection system may be summarized as including a detector unit wearable by a user, including: a power input coupleable to a lighting device to provide power from the lighting device to the detector unit; a status indicator associated with the detector unit; and a controller in electronic communication with the status indicator, the controller including a memory configured to store instructions and at least one processor configured to execute the instructions to detect at least one of an active voltage in an environment in a vicinity of the user, an active electrical current in the environment in the vicinity of the user, and whether a fall event associated with the user has occurred, and change at least one characteristic of the status indicator in response to detecting the at least one of the voltage, the electrical current, and the fall event.

The memory may be configured to store further instructions and the at least one processor may be configured to execute the further instructions to detect whether the voltage is active in the environment in the vicinity of the user, detect whether the electrical current is active in the environment in the vicinity of the user, and detect whether a fall event associated with the user has occurred.

The memory is configured to store further instructions and the at least one processor is configured to execute the further instructions to measure capacitance of the voltage at different amplifications.

The detection system may further include a receiver having a detector unit slot and the detector unit is provided in the form of a functional detector cartridge that is removably insertable in the detector unit slot of the receiver.

The receiver is configured to be removably coupleable to headgear or other equipment worn by the user of the detector unit, the receiver including a latching device within the detector unit slot to selectively secure the functional detector cartridge within the detector unit slot of the receiver.

The receiver is provided in the form of an adapter that is removably coupleable to the lighting device to enable the functional detector cartridge to interface with the lighting device.

The adapter includes an electrical connector for interfacing with an electrical system of the lighting device and at least one electrical contact for interfacing with an electrical system of the functional detector cartridge. The lighting device has a corresponding electrical connector configured to interface with the electrical connector of the adapter when the adapter is secured to the lighting device. The functional detector cartridge includes at least one electrical contact configured to interface with the at least one electrical contact of the adapter to provide power from the lighting device to the functional detector cartridge and/or to enable the functional detector cartridge to receive one or more signals from the lighting device and/or to transmit one or more signals to the lighting device.

The detector unit is in communication with the lighting device and the memory is configured to store further instructions, the at least one processor configured to execute the further instructions to change at least one characteristic of the lighting device in response to detecting the least one of the voltage, the electrical current, and the fall event.

The status indicator includes a plurality of light emitting diodes and/or a speaker.

The memory is configured to store further instructions and the at least one processor is configured to execute the further instructions to change a color of the plurality of light emitting diodes in response to detecting the at least one of the voltage, the electrical current, and the fall event and/or emit different sounds with the speaker in response to detecting the at least one of the voltage, the electrical current, and the fall event, including emitting sounds at different volumes corresponding to a strength of the voltage, a strength of the electrical current, and whether the fall event has occurred.

One or more embodiments of a detection system may be summarized as including: a lighting device; a receiver removably coupleable to the lighting device; and a functional detector cartridge removably received in the receiver, the functional detector cartridge including a controller having a memory configured to store instructions and at least one processor configured to execute the instructions to detect at least one of whether a voltage is active in an environment in a vicinity of a user, whether an electrical current is active in the environment in the vicinity of the user, and whether a fall event associated with the user of the functional detector cartridge has occurred.

The functional detector cartridge includes at least one status indicator in communication with the controller and the memory is configured to store further instructions, the at least one processor configured to execute the further instructions to change at least one characteristic of the at least one status indicator in response to detecting the at least one of the voltage, the electrical current, and the fall event.

The at least one status indicator includes at least one of a plurality of light emitting diodes and a speaker.

The functional detector cartridge is configured to receive power from the lighting device and/or exchange communication signals with the lighting device.

The memory is configured to store further instructions, the at least one processor configured to execute the further instructions to measure capacitance of the voltage at different amplifications.

The receiver is provided in the form of an adapter having a cartridge slot and a latching device within the cartridge slot to selectively secure the functional detector cartridge within the cartridge slot of the adapter.

The receiver is provided in the form of an adapter that is removably coupleable to the lighting device, the adapter including one or more electrical contacts and being configured to support the functional detector cartridge in an electrically connected manner to an electrical system of the lighting device to receive electrical power from the lighting device and/or to enable the functional detector cartridge to receive one or more signals from the lighting device and/or to transmit one or more signals to the lighting device via the one or more electrical contacts of the adapter.

The functional detector cartridge is in communication with the lighting device and the memory is configured to store further instructions with the at least one processor configured to execute the further instructions to change at least one characteristic of the lighting device in response to detecting the at least one of the voltage, the electrical current, and whether the fall event has occurred.

One or more embodiments of a detection system may be summarized as including: a receiver removably coupleable to headgear or other equipment of a user; and a functional detector cartridge removably insertable in the receiver, the functional cartridge including a controller having a memory configured to store instructions and at least one processor configured to execute the instructions to detect at least one of an active voltage in an environment in a vicinity of the user, an active electrical current in the environment in the vicinity of the user, and whether a fall event associated with the user of the functional detector cartridge has occurred.

The functional detector cartridge further includes at least one status indicator in communication with the controller.

The memory is configured to store further instructions and the at least one processor is configured to execute the further instructions to change at least one characteristic of the status indicator in response to detecting the at least one of the voltage, the electrical current, and the fall event.

The at least one status indicator is configured to provide at least one of a visual, audible, and haptic warning to the user.

The memory is configured to store further instructions and the at least one processor is configured to execute the further instructions to measure capacitance of the voltage at different amplifications.

The receiver includes a cartridge slot and a latching device within the cartridge slot to selectively secure the functional detector cartridge within the cartridge slot of the receiver.

The receiver is provided in the form of a clip that is removably coupleable to headgear or other equipment of the user.

One or more embodiments of a detection system may be summarized as including a plurality of detector units in communication with each other and each carryable by a respective user, each of the plurality of detector units including a controller having a memory configured to store instructions and at least one processor configured to execute the instructions to: detect at least one of an active voltage in an environment in a vicinity of the respective user, an active electrical current in the environment in the vicinity of the respective user, and whether a fall event associated with the respective user has occurred; and communicate an alert to one or more other ones of the plurality of detector units and/or to a remote computing device in response to detecting the least one of the voltage field, the current, and the fall event for the respective user.

Each of the plurality of detector units are in communication with a corresponding one of a plurality of external devices, the plurality of external devices having a memory configured to store instructions and at least one processor configured to execute the instructions to display a user interface on the corresponding one of the plurality of external devices to enable the user to configure a respective one of the plurality of detector units, including adjusting a sensitivity of the respective one of the plurality of detector units with respect to the active voltage, the active current, and whether the fall event has occurred.

Each of the plurality of detector units are in communication with a respective one of a plurality of lighting devices, each of the plurality of detector units configured to receive power from the respective one of the plurality of lighting devices and/or to receive one or more signals from the respective one of the plurality of lighting devices and/or to transmit one or more signals to the respective one of the plurality of lighting devices.

The memory of each of the plurality of detector units is configured to store further instructions and the at least one processor of each of the plurality of detector units is configured to execute the further instructions to group subsets of the plurality of detector units together for communication among the detector units in each subset across distinct channels of a communication protocol.

Each of the plurality of detector units includes at least one status indicator and the memory of each of the plurality of detector units is configured to store further instructions, the at least one processor of each of the plurality of detector units configured to execute the further instructions to change at least one characteristic of the status indicator in response to detecting the at least one of the voltage, the current, and the fall event.

In the above description, certain specific details are set forth in order to provide a thorough understanding of various embodiments of the disclosure. However, one skilled in the art will understand that the disclosure may be practiced without these specific details. In other instances, well-known structures associated with detection systems, devices, and methods have not been described in detail to avoid unnecessarily obscuring the descriptions of the embodiments of the present disclosure.

Certain words and phrases used in the specification are set forth as follows. As used throughout this document, including the claims, the singular form “a”, “an”, and “the” include plural references unless indicated otherwise. Any of the features and elements described herein may be singular, e.g., a die may refer to one die. The terms “include” and “comprise,” as well as derivatives thereof, mean inclusion without limitation. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Other definitions of certain words and phrases are provided throughout this disclosure.

The use of ordinals such as first, second, third, etc., does not necessarily imply a ranked sense of order, but rather may only distinguish between multiple instances of an act or a similar structure or material.

Throughout the specification, claims, and drawings, the following terms take the meaning explicitly associated herein, unless the context clearly dictates otherwise. The term “herein” refers to the specification, claims, and drawings associated with the current application. The phrases “in one embodiment,” “in another embodiment,” “in various embodiments,” “in some embodiments,” “in other embodiments,” and other derivatives thereof refer to one or more features, structures, functions, limitations, or characteristics of the present disclosure, and are not limited to the same or different embodiments unless the context clearly dictates otherwise. As used herein, the term “or” is an inclusive “or” operator, and is equivalent to the phrases “A or B, or both” or “A or B or C, or any combination thereof,” and lists with additional elements are similarly treated. The term “based on” is not exclusive and allows for being based on additional features, functions, aspects, or limitations not described, unless the context clearly dictates otherwise. In addition, throughout the specification, the meaning of “a,” “an,” and “the” include singular and plural references.

Generally, unless otherwise indicated, the materials for making the invention and/or its components may be selected from appropriate materials such as composite materials, ceramics, plastics, metal, polymers, thermoplastics, elastomers, plastic compounds, and the like and may include one or more additives, such as light blocking additives or others to reduce transparency or translucence.

The foregoing description, for purposes of explanation, uses specific nomenclature and formula to provide a thorough understanding of the disclosed embodiments. It should be apparent to those of skill in the art that the specific details are not required in order to practice the invention. The embodiments have been chosen and described to best explain the principles of the disclosed embodiments and its practical application, thereby enabling others of skill in the art to utilize the disclosed embodiments, and various embodiments with various modifications as are suited to the particular use contemplated. Thus, the foregoing disclosure is not intended to be exhaustive or to limit the invention to the precise forms disclosed, and those of skill in the art recognize that many modifications and variations are possible in view of the above teachings.

The terms “top,” “bottom,” “upper,” “lower,” “left,” “right,” and other like derivatives are used only for discussion purposes based on the orientation of the components in the Figures of the present disclosure. These terms are not limiting with respect to the possible orientations explicitly disclosed, implicitly disclosed, or inherently disclosed in the present disclosure and unless the context clearly dictates otherwise, any of the aspects of the embodiments of the disclosure can be arranged in any orientation.

As used herein, the term “substantially” is construed to include an ordinary error range or manufacturing tolerance due to slight differences and variations in manufacturing. Unless the context clearly dictates otherwise, relative terms such as “approximately,” “substantially,” and other derivatives, when used to describe a value, amount, quantity, or dimension, generally refer to a value, amount, quantity, or dimension that is within plus or minus 5% of the stated value, amount, quantity, or dimension. It is to be further understood that any specific dimensions of components or features provided herein are for illustrative purposes only with reference to the various embodiments described herein, and as such, it is expressly contemplated in the present disclosure to include dimensions that are more or less than the dimensions stated, unless the context clearly dictates otherwise.

U.S. Provisional Patent Application No. 63/332,188 filed Apr. 18, 2022 is incorporated herein by reference, in its entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the breadth and scope of a disclosed embodiment should not be limited by any of the above-described embodiments, but should be defined only in accordance with the following claims and their equivalents.

1. A detection system, comprising:
a detector unit wearable by a user, including:
a status indicator; and
a controller in electronic communication with the status indicator, the controller including a memory configured to store instructions and at least one processor configured to execute the instructions to:
detect whether a voltage is active in an environment in a vicinity of the user, including measuring capacitance at different amplifications; and
change at least one characteristic of the status indicator in response to detecting the voltage.

2. The detection system of claim 1, wherein the memory is configured to store further instructions, the at least one processor configured to execute the further instructions to:
detect whether an electrical current is active in the environment in a vicinity of the user; and
change at least one characteristic of the status indicator in response to detecting the current.

3. The detection system of claim 1, wherein the memory is configured to store further instructions, the at least one processor configured to execute the further instructions to:
detect whether a fall event associated with the user of the detector unit has occurred; and
change at least one characteristic of the status indicator in response to detecting the occurrence of the fall event.

4. The detection system of claim 1, further comprising:
a receiver having a detector unit slot, wherein the detector unit is provided in the form of a functional detector cartridge that is removably insertable in the detector unit slot of the receiver.

5. The detection system of claim 4, wherein the receiver is configured to be removably coupleable to headgear or other equipment worn by the user of the detector unit and includes a latching device within the detector unit slot to selectively secure the functional detector cartridge within the detector unit slot of the receiver.

6. The detection system of claim 4, further comprising:
a lighting device wearable by the user of the detector unit, wherein the receiver is provided in the form of an adapter that is removably coupleable to the lighting device to enable the functional detector cartridge to interface with the lighting device.

7. The detection system of claim 6, wherein the adapter includes one or more electrical contacts and is configured to support the functional detector cartridge in an electrically connected manner to an electrical system of the lighting device to receive electrical power from the lighting device and/or to enable the functional detector cartridge to receive one or more signals from the lighting device and/or to transmit one or more signals to the lighting device via the one or more electrical contacts of the adapter.

8. The detection system of claim 6, wherein the adapter includes an electrical connector for interfacing with an electrical system of the lighting device and at least one electrical contact for interfacing with an electrical system of the functional detector cartridge, and
wherein the lighting device has a corresponding electrical connector configured to interface with the electrical connector of the adapter when the adapter is secured to the lighting device, and
wherein the functional detector cartridge includes at least one electrical contact configured to interface with the at least one electrical contact of the adapter to provide power from the lighting device to the functional detector cartridge and/or to enable the functional detector cartridge to receive one or more signals from the lighting device and/or to transmit one or more signals to the lighting device.

9. The detection system of claim 6, wherein the functional detector cartridge is in communication with the lighting device, and
wherein the memory is configured to store further instructions, the at least one processor configured to execute the further instructions to:
change at least one characteristic of the lighting device in response to detecting the voltage.

10. The detection system of claim 1, wherein the detector unit is in communication with a lighting device that is remote from the detector unit, and
wherein the memory is configured to store further instructions, the at least one processor configured to execute the further instructions to:
change at least one characteristic of the lighting device in response to detecting the voltage.

11. The detection system of claim 1, wherein the detector unit is configured to receive power from a lighting device to which the detector unit is coupleable.

12. A detection system, comprising:
a detector unit wearable by a user, including:
a status indicator; and
a controller in electronic communication with the status indicator, the controller including a memory configured to store instructions and at least one processor configured to execute the instructions to:
detect whether a voltage is active in an environment in a vicinity of the user;
detect whether an electrical current is active in the environment in the vicinity of the user;
detect whether a fall event associated with the user of the detector unit has occurred; and
change at least one characteristic of the status indicator in response to detecting at least one of the voltage, the electrical current, and the fall event.

13. The detection system of claim 12, further comprising:
a receiver removably coupleable to headgear or other equipment of the user and configured to removably receive the detector unit.

14. The detection system of claim 13, wherein the receiver includes a detector unit slot, the detector unit provided in the form of a functional detector cartridge that is removably insertable in the detector unit slot of the receiver.

15. The detection system of claim 14, wherein the receiver includes a latching device within the detector unit slot to selectively secure the functional detector cartridge within the detector unit slot of the receiver.

16. The detection system of claim 12, wherein the detector unit is configured to receive power from a lighting device in communication with the detector unit.

17. The detection system of claim 12, further comprising:
a lighting device wearable by the user; and
an adapter removably coupleable to the lighting device and configured to removably receive the detector unit to secure the detector unit to the lighting device.

18. The detection system of claim 17, wherein the detector unit is electrically connected to the lighting device, the lighting device configured to provide power to the form factor via the adapter and/or to enable the detector unit to receive one or more signals from the lighting device and/or to transmit one or more signals to the lighting device via the adapter.

19. The detection system of claim 17, wherein the adapter includes an electrical connector for interfacing with an electrical system of the lighting device and at least one electrical contact for interfacing with an electrical system of the functional detector cartridge, and
wherein the lighting device has a corresponding electrical connector configured to interface with the electrical connector of the adapter when the adapter is secured to the lighting device, and
wherein the functional detector cartridge includes at least one electrical contact configured to interface with the at least one electrical contact of the adapter to provide power from the lighting device to the functional detector cartridge and/or to enable the functional detector cartridge to receive one or more signals from the lighting device and/or to transmit one or more signals to the lighting device.

20. The detection system of claim 17, wherein the detector unit is in communication with the lighting device, and
wherein the memory is configured to store further instructions, the at least one processor configured to execute the further instructions to:
change at least one characteristic of the lighting device in response to detecting at least one of the voltage, the electrical current, and the fall event.

21. The detection system of claim 12, wherein the detector unit is in communication with a lighting device, and
wherein the memory is configured to store further instructions, the at least one processor configured to execute the further instructions to:
change at least one characteristic of the lighting device in response to detecting at least one of the voltage, the electrical current, and the fall event.

22. The detection system of claim 12, wherein the memory is configured to store further instructions, the at least one processor configured to execute the further instructions to:
measure capacitance of the voltage at different amplifications.

23. A detection system, comprising:
a detector unit wearable by a user, including:
a power input coupleable to a lighting device or other host device to provide power from the lighting device of other host device to the detector unit;
a status indicator associated with the detector unit; and
a controller in electronic communication with the status indicator, the controller including a memory configured to store instructions and at least one processor configured to execute the instructions to:
detect at least one of an active voltage in an environment in a vicinity of the user, an active electrical current in the environment in the vicinity of the user, and whether a fall event associated with the user has occurred; and
change at least one characteristic of the status indicator in response to detecting the at least one of the voltage, the electrical current, and the fall event.

24. The detection system of claim 23, wherein the memory is configured to store further instructions, the at least one processor configured to execute the further instructions to:
detect whether the voltage is active in the environment in the vicinity of the user;
detect whether the electrical current is active in the environment in the vicinity of the user; and
detect whether a fall event associated with the user has occurred.

25. The detection system of claim 23, wherein the memory is configured to store further instructions, the at least one processor configured to execute the further instructions to:
measure capacitance of the voltage at different amplifications.

26. The detection system of claim 23, further comprising:
a receiver having a detector unit slot, wherein the detector unit is provided in the form of a functional detector cartridge that is removably insertable in the detector unit slot of the receiver.

27. The detection system of claim 26, wherein the receiver is configured to be removably coupleable to headgear or other equipment worn by the user of the detector unit, the receiver including a latching device within the detector unit slot to selectively secure the functional detector cartridge within the detector unit slot of the receiver.

28. The detection system of claim 26, wherein the receiver is provided in the form of an adapter that is removably coupleable to the lighting device to enable the functional detector cartridge to interface with the lighting device.

29. The detection system of claim 28, wherein the adapter includes an electrical connector for interfacing with an electrical system of the lighting device and at least one electrical contact for interfacing with an electrical system of the functional detector cartridge, and
wherein the lighting device has a corresponding electrical connector configured to interface with the electrical connector of the adapter when the adapter is secured to the lighting device, and
wherein the functional detector cartridge includes at least one electrical contact configured to interface with the at least one electrical contact of the adapter to provide power from the lighting device to the functional detector cartridge and/or to enable the functional detector cartridge to receive one or more signals from the lighting device and/or to transmit one or more signals to the lighting device.

30. The detection system of claim 23, wherein the detector unit is in communication with the lighting device, and
wherein the memory is configured to store further instructions, the at least one processor configured to execute the further instructions to:
change at least one characteristic of the lighting device in response to detecting the least one of the voltage, the electrical current, and the fall event.

31. The detection system of claim 23, wherein the status indicator includes a plurality of light emitting diodes and/or a speaker.

32. The detection system of claim 31, wherein the memory is configured to store further instructions, the at least one processor configured to execute the further instructions to:
change a color of the plurality of light emitting diodes in response to detecting the at least one of the voltage, the electrical current, and the fall event; and/or
emit different sounds with the speaker in response to detecting the at least one of the voltage, the electrical current, and the fall event, including emitting sounds at different volumes corresponding to a strength of the voltage, a strength of the electrical current, and whether the fall event has occurred.

33. A detection system, comprising:
a lighting device;
a receiver removably coupleable to the lighting device; and
a functional detector cartridge removably received in the receiver, the functional detector cartridge including a controller having a memory configured to store instructions and at least one processor configured to execute the instructions to:
detect at least one of whether a voltage is active in an environment in a vicinity of a user, whether an electrical current is active in the environment in the vicinity of the user, and whether a fall event associated with the user of the functional detector cartridge has occurred.

34. The detection system of claim 33, wherein the functional detector cartridge includes at least one status indicator in communication with the controller, and
wherein the memory is configured to store further instructions, the at least one processor configured to execute the further instructions to:
change at least one characteristic of the at least one status indicator in response to detecting the at least one of the voltage, the electrical current, and the fall event.

35. The detection system of claim 33, wherein the at least one status indicator includes at least one of a plurality of light emitting diodes and a speaker.

36. The detection system of claim 33, wherein the functional detector cartridge is configured to receive power from the lighting device and/or exchange communication signals with the lighting device.

37. The detection system of claim 33, wherein the memory is configured to store further instructions, the at least one processor configured to execute the further instructions to:
measure capacitance of the voltage at different amplifications corresponding.

38. The detection system of claim 33, wherein the receiver is provided in the form of an adapter having a cartridge slot and a latching device within the cartridge slot to selectively secure the functional detector cartridge within the cartridge slot of the adapter.

39. The detection system of claim 33, wherein the receiver is provided in the form of an adapter that is removably coupleable to the lighting device, the adapter including one or more electrical contacts and being configured to support the functional detector cartridge in an electrically connected manner to an electrical system of the lighting device to receive electrical power from the lighting device and/or to enable the functional detector cartridge to receive one or more signals from the lighting device and/or to transmit one or more signals to the lighting device via the one or more electrical contacts of the adapter.

40. The detection system of claim 33, wherein the functional detector cartridge is in communication with the lighting device, and wherein the memory is configured to store further instructions, the at least one processor configured to execute the further instructions to:
change at least one characteristic of the lighting device in response to detecting the at least one of the voltage, the electrical current, and whether the fall event has occurred.

41. A detection system, comprising:
a receiver removably coupleable to headgear or other equipment of a user; and
a functional detector cartridge removably insertable in the receiver, the functional cartridge including a controller having a memory configured to store instructions and at least one processor configured to execute the instructions to:
detect at least one of an active voltage in an environment in a vicinity of the user, an active electrical current in the environment in the vicinity of the user, and whether a fall event associated with the user of the functional detector cartridge has occurred.

42. The detection system of claim 41, wherein the functional detector cartridge further includes at least one status indicator in communication with the controller.

43. The detection system of claim 42, wherein the memory is configured to store further instructions, the at least one processor configured to execute the further instructions to:
change at least one characteristic of the status indicator in response to detecting the at least one of the voltage, the electrical current, and the fall event.

44. The detection system of claim 41, wherein the at least one status indicator is configured to provide at least one of a visual, audible, and haptic warning to the user.

45. The detection system of claim 41, wherein the memory is configured to store further instructions, the at least one processor configured to execute the further instructions to:
measure capacitance of the voltage at different amplifications.

46. The detection system of claim 41, wherein the receiver includes a cartridge slot and a latching device within the cartridge slot to selectively secure the functional detector cartridge within the cartridge slot of the receiver.

47. The detection system of claim 41, wherein the receiver is provided in the form of a clip that is removably coupleable to headgear or other equipment of the user.

48. A detection system, comprising:
a plurality of detector units in communication with each other and each carryable by a respective user, each of the plurality of detector units including a controller having a memory configured to store instructions and at least one processor configured to execute the instructions to:
detect at least one of an active voltage in an environment in a vicinity of the respective user, an active electrical current in the environment in the vicinity of the respective user, and whether a fall event associated with the respective user has occurred; and
communicate an alert to one or more other ones of the plurality of detector units and/or to a remote computing device in response to detecting the least one of the voltage field, the current, and the fall event for the respective user.

49. The detection system of claim 48, wherein each of the plurality of detector units are in communication with a corresponding one of a plurality of external devices, the plurality of external devices having a memory configured to store instructions and at least one processor configured to execute the instructions to:
display a user interface on the corresponding one of the plurality of external devices to enable the user to configure a respective one of the plurality of detector units, including adjusting a sensitivity of the respective one of the plurality of detector units with respect to the active voltage, the active current, and whether the fall event has occurred.

50. The detection system of claim 48, wherein each of the plurality of detector units are in communication with a respective one of a plurality of lighting devices, each of the plurality of detector units configured to receive power from the respective one of the plurality of lighting devices and/or to receive one or more signals from the respective one of the plurality of lighting devices and/or to transmit one or more signals to the respective one of the plurality of lighting devices.

51. The detection system of claim 48, wherein the memory of each of the plurality of detector units is configured to store further instructions, the at least one processor of each of the plurality of detector units configured to execute the further instructions to:
group subsets of the plurality of detector units together for communication among the detector units in each subset across distinct channels of a communication protocol.

52. The detection system of claim 48, wherein each of the plurality of detector units includes at least one status indicator, the memory of each of the plurality of detector units is configured to store further instructions, and the at least one processor of each of the plurality of detector units configured to execute the further instructions to:
change at least one characteristic of the status indicator in response to detecting the at least one of the voltage, the current, and the fall event.
